(19) United States
(12) Patent Application Publication
(10) Pub. No.: US 2004/0071709 A1
(43) Pub. Date: Apr. 15, 2004

(54) Corona-virus-like particles comprising functionally deleted genomes

(76) Inventors: Petrus Josephus Marie Rottier, Groenekan (NL); Berend-Jan Bosch, Utrecht (NL)

Correspondence Address: TRASKBRITT, PC, P.O. Box 2550, Salt Lake City, UT, 84110, US

(21) Appl. No.: 10/414,256

(22) Filed: Apr. 14, 2003

Related U.S. Application Data
(63) Continuation of application No. PCT/NL02/00318, filed on May 17, 2002.

(30) Foreign Application Priority Data
May 17, 2001 (EP) 01201861.0

(51) Int. Cl. A61K 39/42

(52) U.S. Cl. 424/159.1; 514/2

(57) ABSTRACT

The invention relates to the field of coronaviruses and diagnosis, therapeutics, and vaccines derived thereof. Methods are shown for at least in part inhibiting anti-parallel coiled coil formation of a coronavirus spike protein wherein the methods include decreasing the contact between heptad repeat regions of the protein. The invention provides a peptide including a heptad repeat region of a corona viral spike protein and/or a functional fragment and/or an equivalent thereof. The invention also provides antibodies and inhibiting compounds.

### CROSS-REFERENCE TO RELATED APPLICATION

This application is a continuation in part of PCT International Patent Application No. PCT/NL/02/00318, filed on May 17, 2002, designating the United States of America, and published, in English, as PCT International Publication No. WO 02/092827 A2 on Nov. 11, 2002, the contents of the entirety of which is incorporated by this reference.

### TECHNICAL FIELD

The invention relates generally to biotechnology and medicine and more particularly to the field of coronaviruses and diagnosis, therapeutic use and vaccines derived therefrom.

### BACKGROUND

Coronavirions have a rather simple structure. They consist of a nucleocapsid surrounded by a lipid membrane. The helical nucleocapsid is composed of the RNA genome packaged by one type of protein, the nucleocapsid protein N.

The viral envelope generally has 3 membrane proteins: the spike protein (S), the membrane protein (M), and the envelope protein (E). Some coronaviruses have a fourth protein in their membrane, the hemaglutinin-esterase protein (HE). Like all viruses coronaviruses encode a wide variety of different gene products and proteins. Most important among these are obviously the proteins responsible for functions related to viral replication and virion structure.

However, besides these elementary functions, viruses generally specify a diverse collection of other proteins, the function of which is often still unknown but which are known or assumed to be in some way beneficial to the virus. These proteins may either be essential—operationally defined as being required for virus replication in cell culture—or dispensable.

Coronaviruses constitute a family of large, positive-sense RNA viruses that usually cause respiratory and intestinal infections in many different species. Based on antigenic, genetic and structural protein criteria they have been divided into three distinct groups: group I, I, and III. Actually, in view of the great differences between the groups their classification into three different genera is presently being discussed by the responsible ICTV Study Group. The features that all these viruses have in common are a characteristic set of essential genes encoding replication and structural functions. Interspersed between and flanking these genes sequences occur that differ profoundly among the groups and that are, more or less, specific for each group.

To successfully initiate an infection, viruses need to overcome the cell membrane barrier. Enveloped viruses achieve this by membrane fusion, a process mediated by specialized viral fusion proteins. Most viral fusion proteins are expressed as precursor proteins, which are endoproteolytically cleaved by cellular proteases giving rise to a metastable complex of a receptor binding and a membrane fusion subunit.

### DISCLOSURE OF THE INVENTION

The present invention provides methods and means to interfere with fusion of corona viruses. According to the invention, a receptor binding at the cell membrane, the fusion proteins undergo a dramatic conformational transition. A hydrophobic fusion peptide becomes exposed and inserts into the target membrane. The free energy released upon subsequent refolding of the fusion protein to its most stable conformation is believed not only to facilitate the close apposition of viral and cellular membranes but also to effect the actual membrane merger (1, 46, 54).

The present invention provides methods and means to use the biochemical and functional characteristics of the HR regions of the corona virus spike proteins. We show here that peptides corresponding to the HR regions assemble into a thermostable, oligomeric, alpha-helical rod-like complex, with the HR1 and HR2 helices oriented in an anti-parallel manner.

Furthermore, the invention teaches that HR2 of the corona virus spike protein such as MHV-A59 spike protein is a strong inhibitor of both virus-cell and cell-cell fusion.

The present application also provides the amino acid sequences of the HR regions of a corona virus belonging to another group such as Feline infectious peritonitis (FIP) virus spike protein, and of the inhibition of cell-to-cell fusion in FIPV infected cells by administration of HR2 of viruses such as FIPV. Also demonstrated is that the same mechanism is valid in different groups of coronaviruses.

The present invention also provides the amino acid sequences of the HR regions of the spike protein of a coronavirus which causes a severe acute respiratory syndrome in humans and which has been designated provisionally as sudden severe respiratory syndrome (SARS).

The invention makes use of the discovery that, in coronaviruses, the energy necessary for the membrane fusion process is at least partly provided by the formation of an anti-parallel coiled coil structure by folding of the spike protein and combination of the HR1 and HR2 repeat region.

Decreasing the contact of the heptad repeat regions in the spike protein results in a less optimal fit of the coiled coil and thus in less energy for the fusion of membranes. Therefore, this disclosure teaches a method for at least in part inhibiting anti-parallel coiled coil formation of a coronavirus spike protein comprising decreasing the contact between heptad repeat regions of the protein. Of course, blocking the coiled coil formation by occupying the sequence of either HR1 or HR2 is a good way of decreasing, or even preventing coiled coil formation.

The contact of the heptad repeat regions can be disturbed by a molecule or compound that binds to HR1 or HR2 and by binding to these regions, or in close proximity, the compound blocks the site for binding to another HR site. This will result in decreasing or inhibiting the ability of the coronavirus to fuse with a membrane and enter a cell. Of course, if binding of a compound occurs in the vicinity of these regions, contact of the heptad repeat regions may also be decreased and/or inhibited. Such a compound may for example be a peptide and/or a functional fragment and/or an equivalent thereof with an amino acid sequence as shown in FIG. 1.

A functional fragment of a protein or peptide is defined as a part which has the same kind of biological properties in kind, not necessarily in amount. A functional equivalent of a peptide is defined as a compound be it a peptide or proteinaceous or non-proteinaceous molecule with essentially the same functional properties in kind, not necessarily in amount. A functional equivalent can be provided in many ways, for instance through conservative amino acid substitution.

A person skilled in the art is well able to generate analogous equivalents of a protein. This can for instance be done through screening of a peptide library. Such an equivalent has essentially the same biological properties of the protein or peptide in kind, not necessarily in amount.

Therefore, this disclosure teaches a method for at least in part inhibiting anti-parallel coiled coil formation of a coronavirus spike protein comprising decreasing the contact between heptad repeat regions of the protein, wherein the decreasing is provided by a peptide and/or a functional fragment and/or an equivalent thereof.

Decreasing contact between heptad regions may also be provided by a peptide comprising a heptad repeat region of a coronal spike protein and/or a functional fragment and/or an equivalent thereof. Therefore, the invention includes a method to decrease and/or inhibit contact between heptad regions wherein the decreasing and/or inhibiting is provided by a peptide comprising a heptad repeat region of a coronal spike protein and/or a functional fragment and/or an equivalent thereof. The disclosure of the amino acid sequence of HR2 of SARS enables the production and/or selection of peptides comprising SARS HR2 of spike protein and/or a functional fragment and/or an equivalent thereof

In another embodiment, the decreasing can be achieved by providing an antibody directed against a part of HR1 or HR2. The antibody will inhibit the binding of a heptad repeat region to another heptad repeat region, thus preventing at least in part the formation of an anti-parallel coiled coil. Of course, binding of an antibody to a region in close proximity to the heptad region may also disturb the correct fit of the heptad repeat regions in a coiled coil. Therefore, the present application teaches a method for at least in part inhibiting anti-parallel coiled coil formation of a coronavirus spike protein comprising decreasing the contact between heptad repeat regions of the protein, wherein the decreasing is provided by an antibody and/or a functional fragment and/or an equivalent thereof.

The present application shows comparative data on the amino acid sequences of the HR1 and HR2 region of a number of coronaviruses (FIG. 1) and of SARS coronavirus (FIG. 10). The human coronavirus HCV-229E and the feline infectious peritonitis virus (FIPV), which both belong to the group 1 coronaviruses show an insertion of 14 amino acids in the HR1 and in the HR2 region, which the other coronaviruses like mouse hepatitis virus and another human coronavirus (HCV-OC43) (group 2), and infectious bronchitis virus of poultry (group 3) do not have. This insertion of 14 amino acids in each heptad region may generate more electrostatic power for the fusion of a membrane, once the coiled-coil is formed, because the total length of each heptad alpha helix is elongated by 2 coils. The fact that FIPV and HCV-229E have these extra 2 coils per heptad repeat region may indicate that these viruses need extra energy to fuse the membranes of their host cells. Decreasing this energy by inhibiting at least in part the formation of a coiled coil will effectively decrease the penetrating power of the viruses. Therefore, this disclosure teaches a method for at least in part inhibiting anti-parallel coiled coil formation of a coronavirus spike protein comprising decreasing the contact between heptad repeat regions of the protein, wherein the coronavirus comprises a feline coronavirus and/or a human coronavirus, and/or a mouse hepatitis virus MHV and/or a SARS virus.

After infection of a cell by a coronavirus, the infected cell exhibits coronaviral protein on its surface. Coronaviral spike protein present on the cell membrane surface facilitates the fusion of cell membranes of other cells, thus allowing cell-to-cell fusion and allowing the virus to passage from the infected cell to a neighboring cell without the need to leave the cell. An important step in decreasing viral infection of cells is by preventing the cell-to-cell fusion. By providing a compound such as a peptide or an antibody that decreases and/or inhibits the contact of heptad regions, cell-to-cell fusion will be decreased and/or inhibited. The present invention teaches a method for inhibiting fusion of coronavirus spike protein mediated cell-to-cell fusion, comprising decreasing and/or inhibiting the contact between heptad repeat regions of the spike protein.

The present invention also provides methods for selecting further inhibitors of coiled coil formation in corona viruses. For example, the HR1 and HR2 peptides may be used in vitro to select binding compounds from libraries of molecules. Any compound that binds to at least part of an HR1 or HR2 peptide is selected and is used as an inhibitor of the formation of an anti-parallel coiled coil in a spike protein of coronavirus. Therefore, this application teaches a method to select a binding compound to a heptad repeat region of a coronavirus spike protein, comprising contacting in vitro at least one heptad region of a coronavirus spike protein with a collection of compounds and measuring the formation of an anti-parallel coiled coil in the protein.

The present invention also teaches a compound selected by contacting in vitro at least one heptad region of a coronavirus spike protein with a collection of compounds and measuring the formation of an anti-parallel coiled coil in the protein. With this method, non-proteinaceous compounds, proteinaceous compounds and antibodies are selected for their capacity to bind to the heptad repeat regions. Of course, a functional fragment and/or equivalent of an antibody may also bind to heptad repeat regions. Therefore, this application also teaches an antibody or a functional fragment and/or equivalent thereof, capable of decreasing and/or inhibiting the contact between heptad repeat regions of a coronavirus spike protein. The aforementioned compound and/or antibodies may be incorporated into a pharmaceutical composition with a suitable diluent and/or or carrier compound. Therefore, the application teaches a pharmaceutical composition comprising the compound and/or the antibody or a functional fragment and/or equivalent thereof, and a suitable diluent and/or carrier. Administration of the pharmaceutical composition to a cell or a subject with a corona viral infection will inhibit the infection of cells and at least in part decrease the coronaviral infection. Therefore, the application teaches a method of treatment of coronavirus infections comprising providing to a subject the pharmaceutical composition.

In another embodiment, the compounds and/or antibodies may be used to detect the presence of coronavirus in a cell or in a subject by contacting a sample of the cells or of the subject to the compound or the antibody and visualizing any binding of the coronavirus to the compound and/or the antibody. The visualizing may be performed by any method known in the art, for example by ELISA techniques or by fluorescence or histochemistry. Therefore, the present invention also teaches a diagnostic kit for detecting coronavirus infection in a sample of a subject comprising the compound or the antibody, further comprising a means of detecting binding of the compound or antibody to the coronavirus. In yet another embodiment, the compound may be used to measure antibody titers of a subject. This may be done to diagnose whether a subject is undergoing a coronaviral infection, or has undergone a coronaviral infection in the past. This may be useful, not only for diagnostic purposes, but also for assessing the possible risk of a subject for a coronaviral infection, and for evaluating vaccination efficiency and strategy. Therefore, the present application also teaches a diagnostic kit for detecting coronavirus antibodies in a sample of a subject comprising the compound, further comprising a means of detecting binding of the compound to the antibodies.

In another embodiment of the invention, the amino acid sequence of the heptad repeat regions is manipulated by recombination, insertion, or deletion techniques that are known in the art. Such a manipulation of the coronaviral genome in or around the heptad repeat regions will result decreased and/or inhibited contact of the heptad repeat regions, it will result in attenuation of the coronavirus. Therefore, the invention teaches a method to attenuate a coronavirus comprising decreasing and/or inhibited the contact between heptad repeat regions of the spike protein of the coronavirus. The method enables the production of an attenuated coronavirus with a decreased contact between the heptad repeat regions. Therefore, the invention teaches an attenuated coronavirus characterized in that the contact between heptad repeat regions of the spike protein of the coronavirus is decreased and/or inhibited.

### BRIEF DESCRIPTION OF THE FIGURES

FIG. 1. (A) Schematic representation of the coronavirus MHV-A59 spike protein structure. The glycoprotein has an N-terminal signal sequence (SS) and a transmembrane domain (TM) close to the C-terminus. The protein is proteolytically cleaved (arrow) in an S1 and S2 subunit, which are non-covalently linked. S2 contains two heptad repeat regions (hatched bars), HR1 and HR2, as indicated. (B) Sequence alignment of HR1 and HR2 domains of MHV-A59 with those of HCoV-OC43 (human coronavirus strain OC43), HCoV-229E (human coronavirus strain 229E), FIPV (feline infectious peritonitis virus strain 79-1146) and IBV (infectious bronchitis virus strain Beaudette). HCV-229E and FIPV, MHV-A59 and HCV-OC43 and IBV are representatives of groups 1, 2 and 3, respectively, the three coronavirus subgroups (56). Dark shading marks sequence identity while lighter shading represents sequence similarity. The alignment shows a remarkable insertion of exactly two heptad repeats (14 a.a.) in both HR1 and HR2 of HCV-229E and FIPV, a characteristic of all group 1 viruses. The predicted hydrophobic heptad repeat ‘a’ and ‘d’ residues are indicated above the sequence. The frame shifts in the predicted heptad repeats in HR1 are caused by a stutter (50). Asterisks denote conserved residues, dots represent similar residues. The amino acid sequences of the peptides HR1, HR1a, HR1b, HR1c and HR2 used in this study are presented in italics below the alignments. N-terminal residues derived from the proteolytic cleavage site of the GST-fusion protein are between brackets. A conserved N-glycosylation sequence in the HR2 region is underlined.

FIG. 2. Hetero-oligomeric complex formation of HR1 and HR1a with HR2. (A) HR1 and HR2 on their own or as a preincubated equimolar (80 μM) mix were subjected to 15% tricine SDS-PAGE. Before gel loading, samples were either heated at 100° C. or left at RT. Positions of HR1, HR2 and HR1-HR2 complex are indicated on the left, while the positions of molecular mass markers are indicated at the right. (B) Same as (A) but with peptide HR1a instead of HR1.

FIG. 3. Temperature stability of HR1-HR2 complex. An equimolar mix of HR1 and HR2 (80 μM) was incubated at RT for 1 h. Samples were subsequently heated for 5 min at the indicated temperatures in 1× tricine sample buffer and analyzed by SDS-PAGE in a 15% tricine gel, together with HR1 and HR2 alone. Positions of HR1, HR2 and HR1-HR2 complex are indicated on the left, while the molecular mass markers are indicated at the right.

FIG. 4. Circular dichroism spectra (mean residue eliplicity) of the HR1 (25 μM; open square) peptide, the HR2 (25 μM; filled triangle) peptide, and of the HR1-HR2 complex (25 μM; filled square) in water at RT. Note that the HR1 and HR2 spectra virtually coincide.

FIG. 5. Electron micrographs of HR1-HR2 complex.

FIG. 6. Proteinase K treatment of HR peptides. The peptides HR2, HR1, HR1 a, HR1b and HR1c were subjected to Proteinase K either individually in solution or after mixing of the different HR1 peptides with HR2 at equimolar concentration followed by a 1 h incubation at 37° C. Proteolytic fragments were separated and purified by HPLC and characterized by mass spectometry. Peptides are schematically indicated by bars. Hatched bars indicate the protease sensitive part(s) of the peptide. N and C-terminal position of the peptide and the amino acid numbering are indicated.

FIG. 7. Inhibition of virus-cell and cell-cell fusion by HR peptides. (A) Virus-cell inhibition by HR peptides using a luciferase gene expressing MHV. LR7 cells were inoculated with virus at an MOI of 5 in the presence of varying concentrations of peptide ranging from 0.4-50 μM. At 5 h p.i. cells were lysed and luciferase activity was measured. (B) Inhibition of spike mediated cell-cell fusion by HR peptides. BSR T7/5 effector cells—BHK cells constitutively expressing T7 RNA polymerase (3), were infected with vaccinia virus for 1 h and subsequently transfected with a plasmid containing the S gene under a T7 promotor. Three hours post transfection, LR7 target cells transfected with a plasmid carrying the luciferase gene behind a T7 promoter, were added to the effector cells. Cells were incubated for another 4 h in the presence or absence of HR peptide. Cells were lysed and luciferase activity was measured.

FIG. 8. Schematic representation (approximately to scale) of the viral fusion proteins of six different virus families; MHV-A59 S (Coronaviridae), Influenza HA (Orthomyxoviridae), HIV-1 gp160 (Retroviridae), SV5 F, (Paramyxoviridae), Ebola Gp2 (Filoviridae) and SeMNPV F (Baculoviridae). Cleavage sites are indicated by triangles; the black bars represent the (putative) fusion peptides, the vertically hatched bars the HR1 domains and the horizontally hatched bars the HR2 domains. Transmembrane domains are indicated by the vertical, dashed lines. For each polypeptide the total length is given at the right.

FIG. 9. GST-FIPV fusion protein sequences of HR1 and HR2.

FIG. 10. (A) Schematic representation of the coronavirus MHV-A59 spike protein structure. The glycoprotein has an N-terminal signal sequence (SS) and a transmembrane domain (TM) close to the C-terminus. The protein is proteolytically cleaved (arrow) in an S1 and S2 subunit, which are non-covalently linked. S2 contains two heptad repeat regions (hatched bars), HR1 and HR2, as indicated. (B) Sequence alignment of HR domains of MHV-A59 with those of HCoV-OC43 (human coronavirus strain OC43), HCoV-229E (human coronavirus strain 229E), FIPV (feline infectious peritonitis virus strain 79-1146) and IBV (infectious bronchitis virus strain Beaudette) and the SARS-associated coronavirus. The alignment shows a remarkable insertion of exactly two heptad repeats (14 a.a.) in both HR1 and HR2 of HCV-229E and FIPV, a characteristic of all group 1 viruses. The predicted hydrophobic heptad repeat ‘a’ and ‘d’ residues are indicated above the sequence. Asterisks denote conserved residues, dots represent similar residues. Note that the numbering of the amino acid sequence of the SARS-associated coronavirus refers to the amino acid sequence as deduced from the sequenced RT-PCR fragment from this virus. The amino acid sequences of the peptides HR1, HR1a, HR1b, HR1c and HR2 used in this study are presented in italics below the alignments. N-terminal residues derived from the proteolytic cleavage site of the GST-fusion protein are between brackets. A conserved N-glycosylation sequence in the HR2 region is underlined.

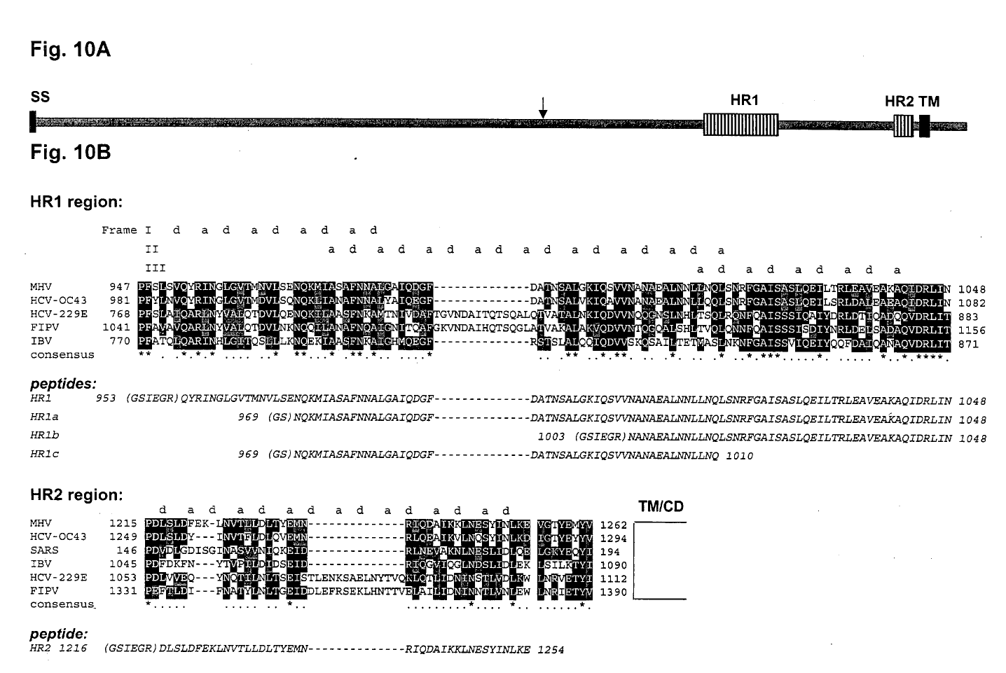

FIG. 11 SARS nucleotide and deduced protein sequence as derived from the RT-PCR fragment.

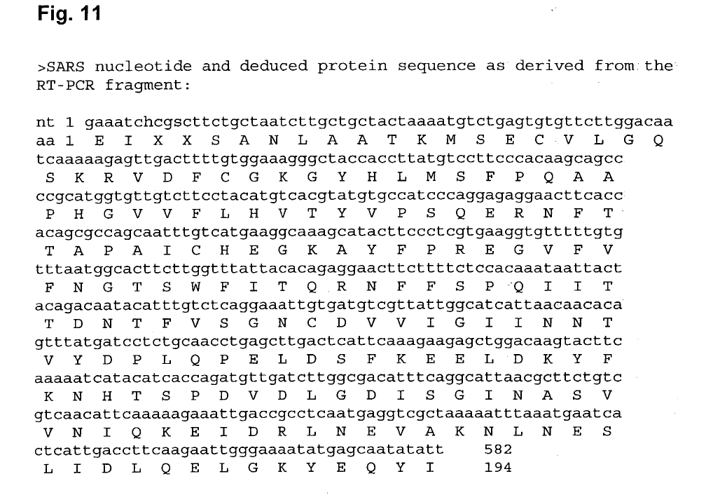

### DETAILED DESCRIPTION OF THE INVENTION

For polyclonal antisera, the peptides or antigens may, if desired, be coupled to a carrier protein, such as KLH as described in Ausubel et al, supra. The KLH-peptide is mixed with Freund's adjuvant and injected into guinea pigs, rats, goats or preferably rabbits. Antibodies may be purified by any method of peptide antigen affinity chromatography.

Alternatively, monoclonal antibodies may be prepared using a SARS polypeptide (or immunogenic fragment or analog) and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In: Monoclonal Antibodies and T Cell Hybridomas, Elsevier, N.Y., 1981; Ausubel et al., supra).

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fe fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

In addition antibody fragments which contain specific binding sites for SARS peptides and antigens may be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments: Alternatively, Fab expression libraries may be constructed to allow rapid and easy identification of monoclonal Fab fragments with the desired specificity (Huse, W. D. et al (1989) Science 256:1275-1281).

Once produced, the polyclonal or monoclonal antibody is tested for specific recognition by Western blot or immunoprecipitation analysis (by the methods described in Antibodies: A Laboratory Manual, (eds. E. Harlow and D. Lane, Cold Spring Harbor, N.Y., 1988)). Lysis and fractionation of SARS protein-harboring cells prior to affinity chromatography may be performed by standard methods (see, e.g., Antibodies: A Laboratory Manual, supra). In another example, an anti-SARS protein antibody (for example, produced as described herein) may be attached to a column and used to isolate the SARS protein.

The compositions can be used for example as targets in combinatorial chemistry protocols or other screening protocols to isolate molecules that possess desired functional properties related to, for example, SARS antigens. The disclosed compositions or antibodies can be used as either reagents in micro arrays or as reagents to probe or analyze existing microarrays. The disclosed compositions can be used in any known method for isolating or identifying SARS related antigens or polypeptides. Alternatively, the compositions can be used in any known method for isolating or identifying SARS related antibodies, for example by detecting the presence of SARS antibodies in a sample. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

The compositions can be used for example as targets in combinatorial chemistry protocols or other screening protocols to isolate molecules that possess desired functional properties related to, for example, SARS antigens. The disclosed compositions or antibodies can be used as either reagents in micro arrays or as reagents to probe or analyze existing microarrays. The disclosed compositions can be used in any known method for isolating or identifying SARS related antigens or polypeptides. Alternatively, the compositions can be used in any known method for isolating or identifying SARS related antibodies, for example by detecting the presence of SARS antibodies in a sample. The compositions can also be used in any known method of screening assays, related to chip/micro arrays.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, a SARS gene product, or homologs and ortholog gene products or fragments of the same are used as targets, or when they are used in competitive inhibition assays are also disclosed. Thus, the products produced using the combinatorial or screening approaches that involve the disclosed compositions are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS. 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference).

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636, which are herein incorporated by reference.

A large number of methods exist to detect the binding or interaction of two or more molecules, including, but are not limited to, immunoprecipitation (Kang et al. (1997) Mol. Cells, 7:237-243; Gharbia et al. (1994) J. Peridontol. 65:56-61), immunohistology (Navarro et al., (1998) Neurosci. Lett. 254:17-20; Nitta et al. (1993) Biol. Reprod. 48:110-116; Heider and Schroeder, (1997) J. Virol. Methods, 66:311-316), immunoblotting (Beesley, J. E., Immunochemistry: A Practical Approach (IRL Press, Oxford, England, 1993), ELISA (Macri and Adeli (1993) B. Eur. J. Clin. Chem. Clin. Biochem. 31:441-446; Rodriguez et al. (1990) J. Dairy Res. 57:197-205), immunoelectrophoresis, immunofluorescence (Avarameas et al (1978) J. Immunol. 8, suppl. 7:7; Wilson and Nakane, Immunofluorescence and Related Staining Techniques, p215 (Elsevier/North Holland Biomedical Press Amsterdam, 1978), chromatography (for example, chromatography may use denaturing and/or non-denaturing conditions, and my involve, the use of any kind of resin, such as, Nickel Affinity, hydroxyapatite, silica, amino acids, carbohydrate binding matrices, carbohydrate matrices, chelating resins, ion exchange, anion exchange, HPLC, Liquid chromatography, immunoaffinity matrices and other specialized resins), western blotting, far western blotting, radioisotope labeling, luciferase assays, two-hybrid based assays (numerous two-hybrid based assays systems are commercially available), Phage display assays, chemiluminescence assays and/or fluorescence assays. The molecules may be labeled or detected with radioisotopes (for example, ³², ³H, ¹³C and/or ¹²⁵I), Biotin Fluorescent molecules (for example,CY3, CY5, Fluorescein, DAPI, R-PPhycoerythrin, PKH2, PKH26, PKH67, Propidium Iodide, Quantum Red™, Rhodamine, Texas Red or others known in the art), Protein G, or A (which bind the Fe region of many mammalian IgG molecules) or protein L (which binds to the kappa light chains of various species), gold (for example, colloidal gold) and/or enzymes (Preferably SARS peptides or antigens, where desirable and appropriate, are “tagged” with an epitope having available one or more antibodies or molecules which specifically bind (commercially available antibodies, specific to enzymes, molecules and epitope tags, are well known in the art)). A molecule having a “tag” (Pretorius et al. (1997) Onderstepoort J. Vet. Res. 64:201-203), includes, but not limited to, myc-, HA-, GST-, V-5-, Lex-A-, cI-, DIG-, Maltose binding protein-, Cellulose binding domain-, streptavidin, Alkaline phosphatase (O'Sullivan et al. (1978) FEBS Lett. 95:311-313), Horseradish Peroxidase, green fluorescent protein, 3×FLAG®-, HIS-Select™-, EZView™—S-Gal™-tags (available from Sigma, Life Science Research).

With a positive stranded RNA genome of 28-32 kb, the Coronaviridae are the largest enveloped RNA viruses. Coronaviruses exhibit a broad host range, infecting mammalian and avian species. They are responsible for a variety of acute and chronic diseases of the respiratory, hepatic, gastrointestinal and neurological systems (56). Recently, coronavirus induced pneumonia (Severe Acute Respiratory Syndrome or “SARS”) has spread rapidly from China via Hong Kong to the rest of the world. The spike (S) protein is the sole viral membrane protein responsible for cell entry. It binds to the receptor on the target cell and mediates subsequent virus-cell fusion (6). Spikes can be seen under the electron microscope as clear, 20 nm large, bulbous surface projections on the virion membrane (14). The spike protein of mouse hepatitis virus (MHV-A59) is a 180 kDa heavily N-glycosylated type I membrane protein which occurs in a homodimeric (37, 66) or homotrimeric (16) complex. In most murine hepatitis strains, the S protein is cleaved intracellularly into an N-terminal subunit (S1) and a membrane anchored subunit (S2) of similar size, which are non-covalently linked and have distinct functions. Binding to the MHV receptor (MHVR) (74) has been mapped to the N-terminal 330 amino acids (a.a.) of the S1 subunit (62), whereas the membrane fusion function resides in the S2 subunit (78). It has been suggested that the S1 subunit forms the globular head while the S2 subunit constitutes the stalk-like region of the spike (15). Binding of S1 to soluble MHVR, or exposure to 37° C. and an elevated pH (pH 8.0) induces a conformational change which is accompanied by the separation of S1 and S2 and which might be involved in triggering membrane fusion (21, 27, 60). Cleavage of the S protein into S1 and S2 has been shown to enhance fusogenicity (25, 61) but cleavage is not absolutely required for fusion (2, 26, 59, 61).

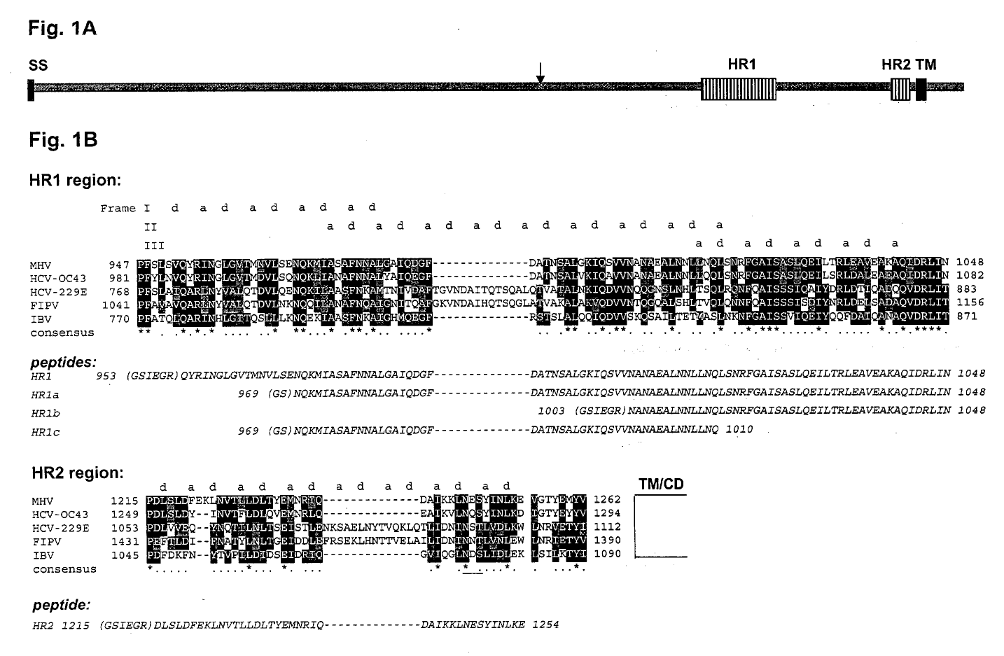

The ectodomain of the S2 subunit contains two regions with a 4,3 hydrophobic (heptad) repeat (15), a sequence motif characteristic of coiled coils. These two heptad repeat (HR) regions, designated here as HR1 and HR2, are conserved in position and sequence among the members of the three coronavirus antigenic clusters (FIG. 1). A number of studies have shown that the HR1 and HR2 regions are involved in viral fusion. First, a putative internal fusion peptide has been proposed to occur close to (7) or within (40) the HR1 region. Second, viruses with mutations in the membrane-proximal HR2 region exhibited defects in spike oligomerization and in fusion ability (39). Third, it has been suggested that the MHV-4 (JHM) strain can utilize both endosomal and nonendosomal pathways for cell entry but does not require acidification of endosomes for fusion activation (48). However, mutations found in murine hepatitis viruses which do require a low pH for fusion, appeared to map to the HR1 region (23).

HR regions appear to be a common motif in many viral fusion proteins (57). There are usually two of them; one N-terminal HR region (HR1) adjacent to the fusion peptide and a C-terminal HR region (HR2) close to the transmembrane anchor. Structural studies on viral fusion proteins reveal that the HR regions form a six-helix bundle structure implicated in viral entry (reviewed in (18)). The structure consists of a homotrimeric coiled coil of HR1 domains in the exposed hydrophobic grooves of which the HR2 regions are packed in an anti-parallel manner. This conformation brings the N-terminal fusion peptide in close proximity to the transmembrane anchor. Because the fusion peptide inserts into the cell membrane during the fusion event, such a conformation facilitates a close apposition of the cellular and viral membrane (reviewed in (18)). Recent evidence suggests that the actual six-helix bundle formation is directly coupled to the merging of the membranes (46, 54). The similarities in the structures of the six-helix bundle complexes elucidated for influenza virus HA (4, 11), human and simian immunodeficiency virus (HIV-1, SIV) gp41 (5, 8, 41, 63, 69, 76), Moloney murine leukemia virus type 1 (MoMLV) gp21 (19), Ebola virus GP2 (42, 68), human T-cell leukemia virus type I (HTLV-1) gp21 (32), Visna virus TM, (43), simian parainfluenza virus (SV5) F1 (1), and human respiratory syncytial virus (HRSV) F1(80), all point to a common fusion mechanism for these viruses.

Based on structural similarities, two classes of viral fusion proteins have been distinguished (36). Proteins containing HR regions and an N-terminal or N-proximal fusion peptide are classified as class I viral fusion proteins. Class II viral fusion proteins (e.g., the alphavirus E1 and the flavivirus E fusion protein) lack HR regions and have an internal fusion peptide. Their fusion protein is folded in tight association with a second protein as a heterodimer. Here, fusion activation takes place upon cleavage of the second protein.

The coronavirus fusion protein (S) shares several features with class I virus fusion proteins. It is a type I membrane protein, synthesized in the ER, and is transported to the plasma membrane. It contains two heptad repeat sequences, one located downstream of the fusion peptide and one in close proximity to the transmembrane region.

However, despite its similarity to class I fusion proteins, there are several characteristics that make the coronavirus S protein exceptional. One is the absence of an N-terminal or even N-proximal fusion peptide in the membrane-anchored subunit. Another peculiarity is the relatively large sizes of the HR regions (˜100 and ˜40 a.a.). Third, cleavage of the S protein is not required for membrane fusion; rather, it does not occur at all in the group 1 coronaviruses. For these reasons, it is not likely to assume that coronavirus fusion protein is a class 1 fusion protein.

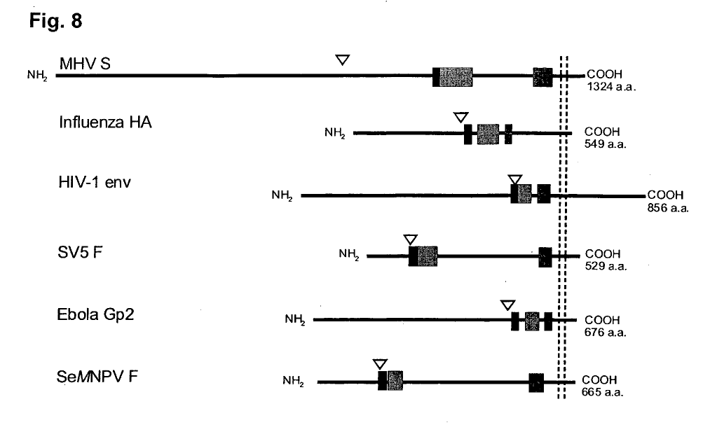

Heptad repeat regions play an important role in viral membrane fusion. Fusion proteins from widely disparate virus families have been shown to contain two such regions, one located close to the fusion peptide, the other generally in the vicinity of the viral membrane ((7); summarized in FIG. 8). Distances between the HR regions vary greatly, from some 50 a.a. as in HIV-1 to about 300 residues in Spodoptera exigua multicapsid nucleopolyhedrosis virus (71). The crystal structures resolved for influenza HA (4, 10, 75) HIV-1 and SIV gp41 (5, 8, 41, 63, 69, 76), MuMLV gp21 (19), Ebola virus GP2 (42, 68), HTLV-1 gp21 (32), Visna virus TM, (43), SV5 F1 (1), HRSV F1 (80) and NDV F (13) all show a central trimeric coiled coil constituted by three HR1 regions. In some of these structures (e.g. HIV-1 and SIV gp41, SV5 F 1, Ebola virus gp2, Visna virus TM and HRSV F1) a second layer of helices or elongated peptide chains was observed contributed by HR2 domains which were packed in an anti-parallel manner into the hydrophobic grooves of the HR1 coiled coil, forming a six-helix bundle. In the full-length protein such a conformation brings the fusion peptide present at the N-terminus of HR1 close to the transmembrane region that occurs at the C-terminal of HR2. With the fusion peptide inserted in the cellular membrane and the transmembrane region anchored in the viral membrane, such a hairpin-like structure facilitates the close apposition of cellular and viral membrane and enables subsequent membrane fusion (reviewed in (18)). Combined with the findings that peptides derived from these HR domains can act as potent inhibitors of fusion (reviewed in (18)), the biological relevance of the heptad repeat regions in the viral life cycle is obvious. Our studies of the heptad repeat motifs in coronavirus spike protein presented here show that coronaviruses use coiled coil formation for membrane fusion and cell entry mechanisms comparable to some other viruses, probably allowing coronavirus spike proteins to be classified as class I viral fusion proteins (36).

The coronavirus (MHV-A59) derived HR peptides exhibited a number of typical class I characteristics. First of all, the purified HR1 and HR2 peptides assembled spontaneously into unique, homogeneous multimeric complexes. These complexes were highly stable surviving, for instance, high concentrations (2%) of SDS and high temperatures (70-80° C.). The peptides apparently associate with great specificity into an energetically very favorable structure. Another typical feature was the observed secondary structure in the peptides. The CD spectra of both the individual and the complexed HR1 and HR2 peptides showed patterns characteristic of alpha-helical structure. Alpha-helix contents were calculated to be about 89% for the separate peptides and about 82% for their equimolar mixture. Consistent with these observations, the HR complex revealed a rod-like structure when examined by electron microscopy. The length of this structure (˜14.5 nm) correlates well with the length predicted for an alpha-helix the size of HR1 (96 a.a.). Similar rod-like structures have been observed for other class I virus fusion proteins such as the influenza virus HA protein (12, 53), portions of the HIV-I gp41 protein (70), and the Ebola virus GP2 protein (67) but the length of the MHV-A59 derived structures is substantially larger. This is presumably even more so for type I coronaviruses which have an insertion of two heptad repeats (14 a.a.; see FIG. 1) in both HR regions. These insertions into otherwise conserved areas suggest these additional sequences to associate With each other in the HR1-HR2 complex thereby extending the alpha-helical complex by exactly four turns. The significance of the exceptional lengths of coronavirus HR complexes may be that the higher energy gain of their formation corresponds with higher energy requirements for membrane fusion by these viruses.

Another important characteristic of class I viral fusion proteins is the formation of a heterotrimeric six-helix bundle during the membrane fusion process, resulting in a close allocation of the fusion peptide and the transmembrane domain. Consistently, protein dissection studies using proteinase K demonstrated an anti-parallel organization of the HR1 and HR2 alpha-helical peptides in the MHV-A59 HR complex. So far, no fusion peptides have been identified in any coronavirus spike protein but predictions for MHV S have located such fusion sequences at (7) or in (40) the N-terminus of HR1. In both cases an anti-parallel orientation of the HR1 and HR2 alpha-helices ensures that the fusion peptide is brought into close proximity to the transmembrane region. Sequence analysis reveals that the ‘e’ and ‘g’ positions in the HR1 regions of all coronaviruses are primarily occupied by hydrophobic residues, unlike the ‘e’ and ‘g’ positions in the HR2 regions which are mostly polar (see FIG. 1). The HR2 region also contains a strictly conserved N-linked glycosylation sequence, indicating its surface accessibility. Preliminary X-ray data on the HR1-HR2 complex show a six-helix bundle structure in the electron dense region (Bosch, B. J., Rottier, P. J. M, and Rey F. A., unpublished results). The combined observations suggest a packing analogous to the fusion proteins of other class I viruses (e.g. HIV, SV5), where the. HR1 and HR2 peptides can form a six-helix bundle with the long HR1 peptide centered in the middle as a three-stranded coiled-coil with the hydrophobic ‘a’ and ‘d’ residues in its inner core. The shorter HR2 peptide packs with its apolar interface in the hydrophobic grooves of the HR1 coiled coil, which expose the mostly hydrophobic residues on ‘e’ and ‘g’ positions.

Peptides derived from the heptad repeat regions of retrovirus (28, 30, 38, 47, 49, 58, 72, 73) and paramyxovirus (29, 35, 51, 77, 79) fusion proteins have been shown to strongly interfere with the fusion activity of these proteins. We observed the same effect when we tested the HR2 peptide of the MHV-A59 spike protein. Using a recombinant luciferase-expressing MHV-A59 the peptide acted as an effective inhibitor of virus entry at micromolar concentrations. Cell-cell fusion inhibition was even more efficiently blocked by the peptide as tested in a cell fusion luciferase assay system. However, peptides derived from the HR1 region had no or only a minor effect on virus entry and syncytia formation. HIV-1 gp41 derived HR peptides that inhibit membrane fusion have been shown not to bind to the native protein or to the six-helix bundle. They can only bind to an intermediate stage of gp41 occurring during the fusion process (9, 20, 31). Repeated passage of HIV in the presence of the inhibitory peptide DP 178, which is derived from the C-terminal gp41 HR region, resulted in resistant viruses containing mutations in the N-terminal HR region (52). Inhibition of membrane fusion by the MHV HR2 peptide most likely takes place during an intermediate stage of the fusion process by binding of the peptide to the HR1 region in the spike protein. This binding, which may occur before, during or after the association of the HR1 regions into the inner trimeric coiled coil, presumably inhibits the subsequent interaction with native HR2 and, consequently, membrane fusion. For the HIV-1 gp41 and SV5 F protein also peptides corresponding to the HR1 region show membrane fusion inhibition, supposedly by binding to the native HR2 region (29, 72). It has been reported previously for HIV-1 that the HR1 peptide aggregates in solution (38) and that its inhibitory activity could be enhanced by fusing it to a designed soluble trimeric coiled coil, making the HR1 peptide more soluble (17). The MHV-A59 HR1 peptide is soluble in water but appeared to precipitate in salt solutions (data not shown). This solubility feature may have obscured the inhibitory potency of our HR1 derived peptides and accounts for the negative results with these peptides in our fusion assays. The HR2 peptide (as well as, soluble forms of HR1) provides powerful antivirals for the therapy of coronavirus induced diseases both in animals and man.

Membrane fusion mediated by class I fusion proteins is accompanied by dramatic structural rearrangements within the viral polypeptide complexes (18). Though little is known of the coronavirus membrane fusion process (for a review, see (22)), the occurrence of conformational changes induced by various conditions has been described for MHV spikes (45). While MHV-A59 is quite stable at mildly acidic pH it is rapidly and irreversibly inactivated at pH 8.0 and 37° C. (60). Under these conditions the S1 subunit dissociates from the virions and the S2 subunit aggregates concomitantly resulting in the aggregation of the particles. Due to the structural rearrangements in the spike, virions can bind to liposomes and the S2 protein becomes sensitive to protease degradation (27). Similar conformational changes can apparently also be induced at pH 6.5 by the binding of spikes to the (soluble) MHV receptor (21, 27) as this interaction enhances liposome binding and protease sensitivity as well (27). Virion binding to liposomes is presumably caused by the exposure of hydrophobic protein surfaces or of the fusion peptide as a result of the conformational change. It appears that the structural rearrangements in the spikes, whether elicited by elevated pH or soluble receptor interaction, reflect the process that naturally gives rise to the fusion of viral and cellular membranes. Accordingly, cell-cell fusion induced by MHV-A59 was maximal at slightly basic pH (60).

A number of studies on the MHV spike protein have shown the importance of the HR regions in membrane fusion. Three codon mutations (Q1067H, Q1094H and L1114R) in or close to the HR1 region of the spike protein were found to be responsible for the low pH requirement for fusion of some MHV-JHM variants isolated from persistently infected cells (23). Analysis of soluble receptor-resistant variants of this virus also pointed to an important role in fusion activity of the HR1 region and suggested that it interacts somehow with the N-terminal domain (S1N330-III; a.a. 278-288) of the spike protein (44). In yet another MHV-JHM. variant a great reduction in cell-cell fusion was attributed to the occurrence of two mutations in the spike protein one of which again located in the HR1 region (A1046V), the other (V870A) in a small non-conserved HR region (N helix) close to the S cleavage site (33). Acidification resulted in a clear enhancement of fusion by this double mutant. It was speculated that the three predicted helical regions (N helix, HR1 and HR2) all collapse into a low-energy coiled-coil during the process of membrane fusion (33). Herein we provide evidence that the HR1 and HR2 regions indeed can form such a low-energy coiled coil. Studies with the MHV-A59 S protein showed that mutations introduced at ‘a’ and ‘d’ positions in an N-terminal part of the HR1 region, a fusion peptide candidate, severely affected cell-cell fusion ability (40). This effect was not due to defects in spike maturation or cell surface expression. Finally, also codon mutations in the HR2 region were found to significantly reduce cell-cell fusion (39). Though these mutant spike protein were apparently impaired in oligomerization their surface expression was hardly affected.

In conclusion, our structural and functional studies show that the coronavirus spike protein can be classified as a class I viral fusion protein. The protein has, however, several unusual features that set it apart. An important characteristic of all class I virus fusion proteins known so far, is the cleavage of the precursor by host cell proteases into a membrane-distal and a membrane-anchored subunit, an event essential for membrane fusion. Consequently, the hydrophobic fusion peptide is then located at or close to the newly generated N-terminus of the membrane anchored subunit, just preceding the HR1 region. In contrast, the MHV-A59 spike does not have a hydrophobic stretch of residues at the distal end of S2, but carries a fusion peptide internally at a location that has yet to be determined (7, 40). Unlike other class I fusion proteins cleavage of the S protein into S1 and S2 has been shown to enhance fusogenicity (25, 61) but not to be absolutely required (2, 26, 59, 61). Rather, spikes belonging to group 1 coronaviruses are not cleaved at all.

The invention is further explained with the aid of the following illustrative examples.

### EXAMPLE I

Materials and Methods

Plasmid constructions. For the production of peptides corresponding to amino acid residues 953-1048 (HR1), 969-1048 (HR1a), 1003-1048 (HR1b), 969-1010 (HR1c) and 1216-1254 (HR2) of the MHV-A59 spike protein, PCR fragments were prepared using as a template the plasmid pTUMS which contains the MHV-A59 spike gene (64). Primers were designed (see Table 1) to introduce into the amplified fragment an upstream BamHI site, a downstream EcoRI site as well as a stop codon preceding the EcoRI site. The fragments corresponding to a.a. 953-1048 and 1216-1254 were additionally provided with sequences specifying a factor Xa cleavage site immediately downstream the BamHI site. Fragments were cloned into the BamHI/EcoRI site of the pGEX-2T bacterial expression vector (Amersham Bioscierice) in frame with the GST gene just downstream of the thrombin cleavage site.

TABLE 1
Primers used for PCR of HR regions

| Primer | Polarity | Sequence (5′-3′) | HR product |
|---|---|---|---|
| 973 | + | GTGGATCCATCGAAGGTCGTCAAT | HR1 |
| | | ATAGAATTAATGGTTTAG | |
| | | (SEQ ID NO:_) | |
| 974 | + | GTGGATCCATCGAAGGTCGTAATG | HR1b |
| | | CAAATGCTGAAGC | |
| | | (SEQ ID NO:_) | |
| 975 | − | GGAATTCAATTAATAAGACGATCT | HR1, HR1a, |
| | | ATCTG | HR1b |
| | | (SEQ ID NO:_) | |
| 976 | − | CGAATTCATTCCTTGAGGTTGATG | HR2 |
| | | TAG | |
| | | (SEQ ID NO:_) | |
| 990 | + | GCGGATCCATCGAAGGTCGTGATT | HR2 |
| | | TATCTCTCGATTTC | |
| | | (SEQ ID NO:_) | |
| 1151 | + | GTGGATCCAACCAAAAGATGATTG | HR1a, HR1c |
| | | C | |
| | | (SEQ ID NO:_) | |
| 1152 | − | GGAATTCAATTGAGTGCTTCAGCA | HR1c |
| | | TTTG | |
| | | (SEQ ID NO:_) | |

To establish a cell-cell fusion inhibition assay, the firefly luciferase gene was cloned under a T7 promoter and an EMCV IRES. The luciferase gene containing fragment was excised from the pSP-luc+vector (Promega) by digestion with NcoI and EcoRV, treated with Klenow, and ligated into the BamHI-linearized, Klenow-blunted pTN3 vector (65) yielding the pTN3-luc+reporter plasmid.

Bacterial protein expression and purification. Freshly transformed BL21 cells (Novagen) were grown in 2×YT (yeast-tryptone) medium to log phase (OD600˜1.0) and subsequently induced by adding IPTG (GibcoBRL) to a final concentration of 0.4 mM. Two hours later cells were pelleted, resuspended in 1/25 volume of 10 mM Tris (pH 8.0), 10 mM EDTA, 1 mM PMSF and sonicated on ice (5 times 2 min). Cell homogenates were centrifuged at 20,000×g for 60 min at 4° C. To each 50 ml of supernatant 2 ml glutathione-sepharose 4B (Amersham Bioscience; 50% v/v in PBS) was added and incubated overnight (O/N) at 4° C. under rotation. Beads were washed three times with 50 ml PBS and resuspended in a final volume of 1 ml PBS. Peptides were cleaved from the GST moiety on the beads using 20 U of thrombin (Amersham Bioscience) by incubation for 4 h at room temperature (RT). Peptides in the supernatant were purified by high pressure reversed phase chromatography (RP-HPLC) using a Phenyl-5PW RP column (Tosoh) with a linear gradient of acetonitrile containing 0.1% trifluoroacetic acid. Peptide containing fractions were vacuum-dried O/N and dissolved in water. Peptide concentration was determined by measuring the absorbance at 280 nm (24) and by BCA protein analysis (Micro BCA™ Assay Kit, Pierce).

Temperature stability of HR1-HR2 complex. An equimolar mix of peptides HR1 and HR2 (80 μM each) in H₂O was incubated at RT for 1 h. After addition of an equal volume of 2×tricine sample buffer (0.125 M Tris pH 6.8, 4% SDS, 5% β-mercaptoethanol, 10% glycerol, 0.004 g bromophenol blue) (55), the mixtures were either left at RT or heated for 5 min at different temperatures and subsequently analyzed by SDS-polyacrylamide gel electrophoresis (PAGE) in 15% tricine gel (55).

CD spectroscopy. CD spectra of peptides (25 μM in H₂O) were recorded at RT on a Jasco J-810 spectropolarimeter, using a 0.1 mm path length, 1 nm bandwidth, 1 nm resolution, 0.5 s response time and a scan speed of 50 nm/min. The alpha-helix content was calculated using the program CDNN (http://bioinformatik.biochemtech.uni-halle.de/cd_spec/).

Electron Microscopy. A preincubated equimolar mix of the peptides HR1 and HR2 was subjected to size-exclusion chromatography (Superdex™ 75 HR 10/30, Amersham Pharmacia Biotech). A sample from the HR1-HR2 peptide complex containing fraction was adsorbed onto a discharged carbon film, negatively stained with a 2% uranyl acetate solution and examined with a Philips CM200 microscope at 100 kV.

Proteinase K treatment. Stock solutions (1 mM) of the peptides HR1, HR1a, HR1b, HR1c and HR2 in water were diluted to 80 μM in PBS. Peptides on their own (80 μM) or after preincubation for 1 h at 37° C. with HR2 (80 μM each) were subsequently subjected to proteinase K digestion (1% wt/wt, proteinase K/peptide) for 2 h at 4° C. Samples were immediately subjected to tricine SDS-PAGE analysis. Protease resistant fragments were also separated and purified by RP HPLC and characterized by mass spectrometry.

Virus-cell fusion assay. The potency of HR peptides in inhibiting viral infection was determined using a recombinant MHV-A59, MHV-EFLM that expresses the firefly luciferase gene (C. A. M. de Haan and P. J. M. Rottier, manuscript in preparation). LR7 cells (34) were maintained as monolayer cultures in Dulbecco's modified Eagle's medium (DMEM) supplemented with 10% fetal calf serum (FCS; GIBCO BRL). LR7 cells grown in 96-wells plates were inoculated with MHV-EFLM in DMEM at a multiplicity of infection (MOI) of 5 in the presence of varying concentrations of peptide ranging from 0.4-50 μM. After 1 h, cells were washed with DMEM and medium was replaced with DMEM containing 10% FCS. At 5 h post infection (p.i.) cells were harvested in 50 μl 1× Passive Lysis buffer (Luciferase Assay System, Promega) according to the manufacturer's protocol. Upon mixing of 10 μl cell lysate with 40 μl substrate, luciferase activity was measured using a Wallac Betalumino meter.

Cell-cell fusion assay. 2×10⁶ LR7 cells, used as target cells, were washed with DMEM and overlayed with transfection medium consisting of 0.2 ml DMEM containing 10 μl of lipofectin (Life Technologies) and 4 μg of the plasmid pTN3-luc+. After 10 min at RT, 0.8 ml DMEM was added and incubation was continued at 37° C. BSR T7/5 cells—BHK cells constitutively expressing T7 RNA polymerase (3); a gift from Dr. K. K. Conzelmann—were grown in BHK-21 medium supplemented with 10% FCS, 100 IU of penicillin/ml and 1 mg/ml geneticin (GIBCO BRL). 1×10⁴ BSR T7/5 cells, designated as effector cells, were infected in 96-wells plates with wild-type vaccinia virus at an MOI of 1 in DMEM at 37° C. After 1 h, the cells were washed with DMEM and incubated for 3 h at 37° C. with transfection medium consisting of 50 μl DMEM containing 1 μl lipofectin and 0.2 μg of the plasmid pTUMS (65), which carries the MHV-A59 spike gene under the control of a T7 promoter. Then, 3×10⁴ of target cells in 100 μl DMEM were added and the cells were incubated for another 4 h in the presence or absence of HR peptide. Cells were lysed and luciferase activity was measured as mentioned above.

Results

HR1 and HR2 Regions in Coronavirus Spike Proteins.

The S2 subunit ectodomain of coronaviruses contains two heptad repeat domains HR1 and HR2, which are conserved in sequence and position (15) (diagrammed in FIG. 1A). HR2 is located adjacent to the transmembrane domain while HR1 occurs at about 170 a.a. upstream of HR2. FIG. 1B shows a protein sequence alignment of the HR1 and HR2 regions for 5 coronaviruses from the three antigenic clusters. The sequence alignment reveals a remarkable insertion of exactly two heptad repeats (14 a.a.) in both the HR1 and the HR2 domain of the spike protein of the group 1 coronaviruses HCV-229E (human coronavirus strain 229E) and FIPV (feline infectious peritonitis virus strain 79-1146). Alignment of all known coronavirus spike protein sequences shows these insertions in all group 1 coronaviruses. Another characteristic feature is that the length of the linker region between the HR2 region and the transmembrane region is strictly conserved in all coronavirus spike proteins.

HR1 and HR2 can Form an Hetero-Oligomeric Complex.

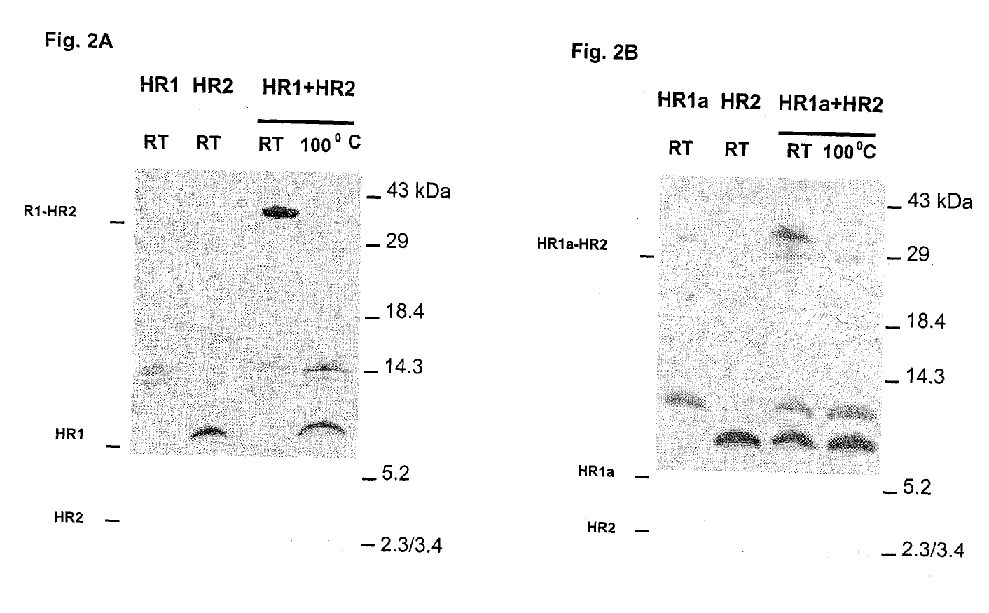

To study the heptad repeat regions in the S2 subunit of MHV-A59, peptides corresponding to the heptad repeat residues 953-1048 (HR1), 969-1048 (HR1a), 969-1048 (HR1b), 969-1003 (HR1c) and 1216-1254 (HR2) (FIG. 1B) were produced in bacteria as GST fusion proteins. Peptides were affinity purified using glutathione-sepharose beads, proteolytically cleaved from the resin and purified to homogeneity by reversed-phase HPLC. Masses of the peptides, as determined by mass spectrometry, matched their predicted Mw (HR1, 10,873 Da; HR1a, 8,653 Da; HR1b, 5,631 Da; HR1c, 4,447 Da; and HR2, 5,254 Da). To study an interaction between the two HR regions, the purified peptides HR1 and HR2 were incubated alone (80 μM) or in an equimolar (80 μM each) mixture for 1 h at 37° C. and the samples were subjected to SDS-PAGE either directly or after heating for 5 min at 95° C. (FIG. 2A). While the peptides migrated according to their molecular weight after separate incubation, most of the protein of the preincubated mixture of HR1 and HR2-migrated as a higher molecular weight complex with a slightly lower mobility than the 29 kDa marker. Upon heating, the complex dissociated giving rise to the individual subunits HR1 and HR2. We also tested the other HR1 peptides for interaction with HR2. While we did not observe complexes upon mixing of HR2 with HR1b or HR1c (data not shown), a higher molecular weight species co-migrating with the 29 kDa marker was found when HR1a was incubated with HR2 (FIG. 2B), though the extent of complex formation appeared to be lower than with peptide HR1. Higher molecular weight species were not seen. The results indicated that the HR1 region contains the information to associate with the HR2 region into a hetero-oligomeric complex and that this complex was stable in the presence of 2% SDS.

HR1-HR2 Complex is Highly Temperature Resistant.

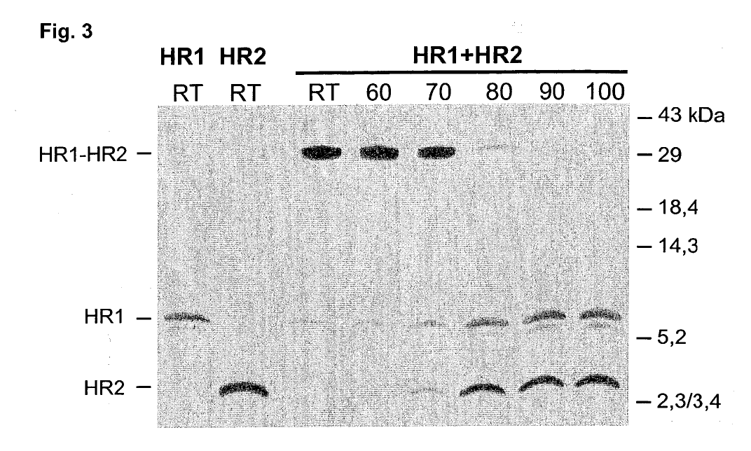

Next we determined the stability of the HR1-HR2 complex at increasing temperatures. An equimolar (80 μM each) mix of the two peptides was again incubated for 1 h at 37° C. and subsequently heated for 5 minutes at different temperatures in 1× tricine sample buffer or left at RT. The complexes were analyzed by SDS-PAGE in 15% gel. As FIG. 3 demonstrates, the high molecular weight complexes remained intact up to 70° C., dissociated partly at 80° C. and fully at 90° C. The stability of the complex at high temperatures indicates that the peptides are held together by strong interaction forces in an energetically favorable conformation.

HR1, HR2 and the HR1-HR2 Complex are Highly α-Helical.

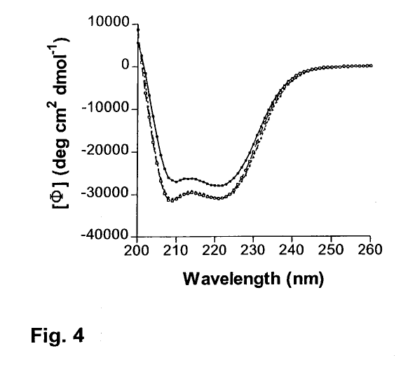

The secondary structure of the HR peptides was examined—by circular dichroism. The CD spectra of HR1, HR2 and of an equimolar mixture of HR1 and HR2 were recorded (FIG. 4). The spectra showed clear minima at 208 nm and 222 nm, which is characteristic of alpha-helical structure. Calculations revealed that the alpha-helical contents of the individual HR1 and HR2 peptides and of the mixture of the two peptides were 89.2%, 89.3% and 81.9%, respectively.

The HR1-HR2 Complex has a Rod-Like Structure.

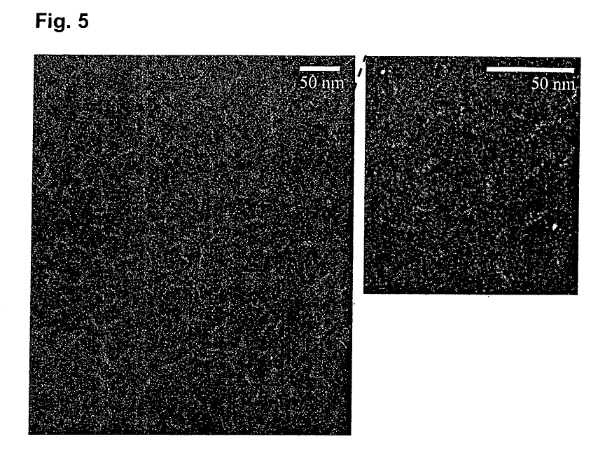

The overall shape of the HR1-HR2 complex was examined by electron microscopy. Complexes were purified and viewed after negative staining. Electron micrographs revealed rod-like structures (FIG. 5). Based on measurements of 40 particles, an average length of 14.5 nm (±2 nm) was calculated. This length is consistent with an alpha-helix of approximately 90 a.a. in length, which corresponds approximately-to the predicted length of the HR1 coiled coil region. Similar rod-shaped complexes have been reported for the influenza virus HA protein (12, 53), for portions of the HIV-1 gp41 protein (70) and for the Ebola virus GP2 protein (67).

HR1 and HR2 Helices Associate in an Anti-Parallel Manner.

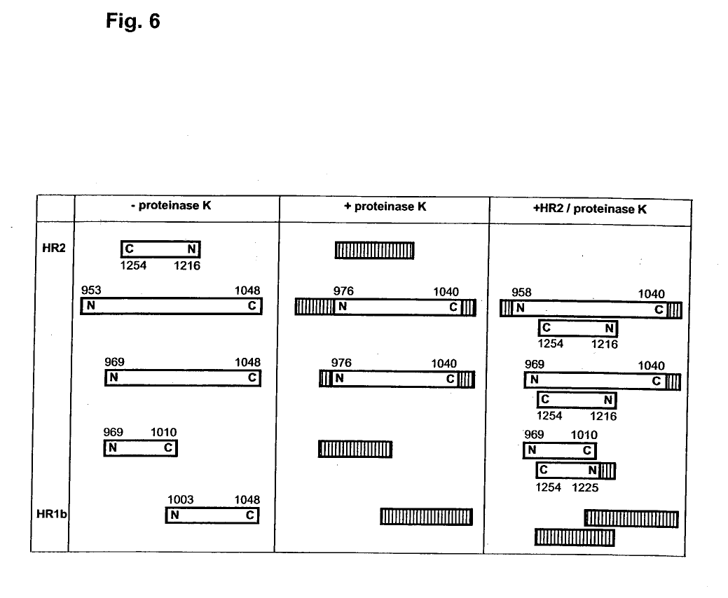

The relative orientation and position of HR2 with respect to HR1 in the complex was examined by limited proteolysis using proteinase K in combination with mass spectrometry. Complexes were generated by incubation of the HR2 peptide with each of peptides HR1, HR1a, HR1b and HR1c. The reaction mixtures as well as the individual peptides were then treated with proteinase K. Samples from each reaction were analyzed by tricine SDS-PAGE (data not shown). Using RP HPLC the protease resistant fragments were purified and their molecular weight (MW) was determined by mass spectrometry, which allowed us to identify the protease resistant cores of the peptides. For each protease resistant core a unique amino acid composition could be deduced that allowed the unequivocal identification of the peptides in the different samples. FIG. 6 gives a schematic overview of the proteinase K resistant fragments. Digestion of HR1 alone left a protease-resistant fragment with a MW of 6,801 Da corresponding to residues 976-1040. Although CD spectra had indicated a folded structure, HR2 was completely degraded by proteinase K. However, in the presence of HR1 HR2 was fully protected from proteolytic degradation. HR2 was able to rescue 18 additional residues at the N terminus of HR1, leaving a fragment of 8,675 Da corresponding to residues 958-1040.

Proteolysis of the HR1a peptide alone generated the same fragment (residues 976-1040) as obtained with HR1. In the HR1a-HR2 mixture, the HR2 peptide was completely protected against degradation by HR1a, while HR2 fully shielded the N-terminus of HR1a for proteolysis, including the glycine and serine residues originating from the thrombin cleavage site.

Although a higher molecular weight species could not be detected by tricine SDS-PAGE (data not shown), the protease treatment of the HR1c-HR2 complex left a protease resistant core. HR1c was fully sensitive for proteinase K, but was completely protected in the presence of HR2. HR2 itself was partly protected against proteolysis by HR1c, yielding a fragment of 3,583 Da that represents residues 1225-1254. Importantly, this HR2 fragment has an intact C-terminus but is degraded at its N-terminus. HR1 c has the same N-terminus as HR1a but is truncated at its C-terminus. Thus, its inability to protect the HR2 N-terminus combined with the full protection provided by HR1a implies an anti-parallel association of the HR1 and HR2 helices in the hetero-oligomeric complex. The peptide HR1b was fully sensitive to proteinase K both by itself and when mixed with HR2. HR1b could not prevent proteolysis of HR2 either. Altogether the proteolysis results suggest the anti-parallel association of HR2 and HR1 to occur in the middle part of HR1.

HR2 Strongly Inhibits Viral Entry and Syncytium Formation.

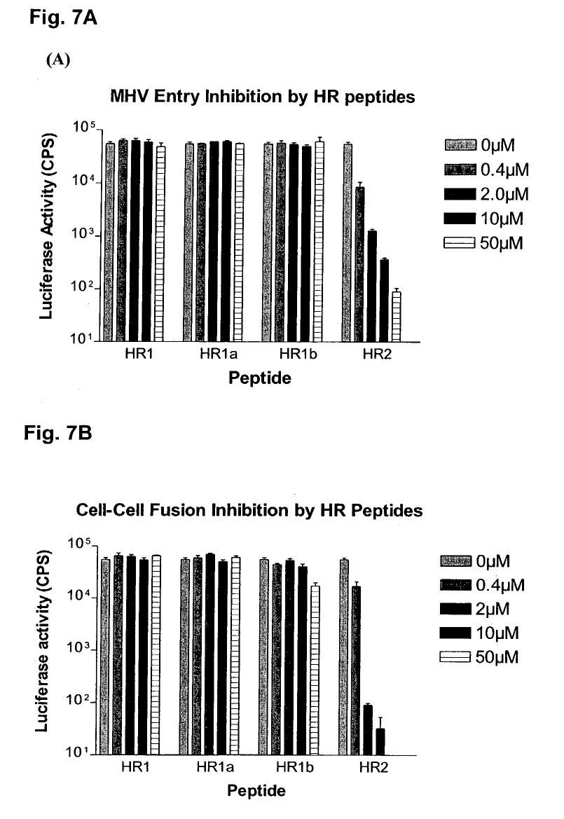

The formation of stable HR complexes is supposedly an essential step in the process of membrane fusion during viral cell entry. Thus, we evaluated the potency of our HR peptides in inhibiting MHV entry making use of a recombinant MHV-A59, MHV-EFLM that expresses the firefly luciferase reporter gene. Cells were inoculated with MHV-EFLM in the presence of different concentrations of the peptides HR1, HR1a, HR1b, HR1c and HR2. After 1 h, the cells were washed and culture medium without peptide was added. At 0.4 h p.i., i.e. before syncytium formation takes place, cells were lysed and tested for luciferase activity (FIG. 7A). HR1, HR1a and HR1b were not able to inhibit virus entry up to concentrations of 50 μM. In contrast, HR2 blocked viral entry in a concentration-dependent, manner inhibition being almost complete at a concentration of 50 μM.

We also studied the ability of the HR peptides in blocking cell-cell fusion. To this end we established a sensitive fusion assay based on the co-culturing of BHK cells expressing the bacteriophage T7 polymerase as well as the MHV-A59 spike protein, with murine L cells transfected with a plasmid carrying a luciferase gene cloned behind a T7 promoter. Fusion of the cells was determined by measuring luciferase activity. The effects of adding the HR peptides during the co-culturing of the cells are compiled in FIG. 7B. The HR2 peptide again appeared to be a potent inhibitor able to efficiently block cell-cell fusion. A 1000× reduction in luciferase activity was measured at a concentration of 10 μM, whereas essentially no activity was observed at a concentration of 50 μM. Of the HR1 peptides only the HR1b peptide had a minor effect at the highest concentration of 50 μM.

### EXAMPLE II

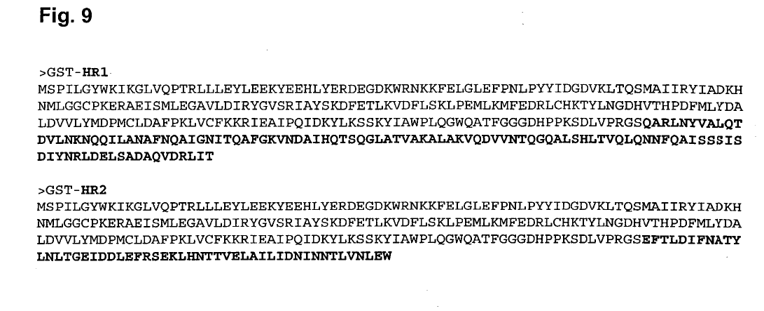

The amino acid sequence of HR1 and HR2 of FIP is shown in FIG. 9.

Inhibition of cell-cell fusion after FIPV infection

FCWF cells were infected with FIPV strain 79-1146 with an moi of 1. 1 hour after infection, the cells were washed and medium was replaced by medium containing the GST-FIPV fusion proteins at different concentrations. 8 hours after infection, cells were fixed and scored for syncytia formation (see, Table 2).

TABLE 2
Inhibition of cell-to-cell fusion
FCFW cells/FIPV infected

| | GST-HR1 | GST-HR2 |
|---|---|---|
| 10 ng | +++ | − |
| 1 ng | +++ | + |
| 0.1 ng | +++ | ++ |
| 0 ng | +++ | +++ |

Syncytia formation +++

### REFERENCES

(The contents of the entirety of all of which are incorporated by this reference).

1. Baker, K. A., R. E. Dutch, R. A. Lamb, and T. S. Jardetzky. 1999. Structural basis for paramyxovirus-mediated membrane fusion. Mol Cell 3:309-19.

2. Bos, E. C., L. Heijnen, W. Luytjes, and W. J. Spaan. 1995. Mutational analysis of the murine coronavirus spike protein: effect on cell-to-cell fusion. Virology 214:453-63.

3. Buchholz, U. J., S. Finke, and K. K. Conzelmann. 1999. Generation of bovine respiratory syncytial virus (BRSV) from cDNA: BRSV NS2 is not essential for virus replication in tissue culture, and the human RSV leader region acts as a functional BRSV genome promoter. J Virol 73:251-9.

4. Bullough, P. A., F. M. Hughson, J. J. Skehel, and D. C. Wiley. 1994. Structure of influenza haemagglutinin at the pH of membrane fusion. Nature 371:37-43.

5. Caffrey, M., M. Cai, J. Kaufman, S. J. Stahl, P. T. Wingfleld, D. G. Covell, A. M. Gronenborn, and G. M. Clore. 1998. Three-dimensional solution structure of the 44 kDa ectodomain of SIV gp41. Embo J 17:4572-84.

6. Cavanagh, D. 1995. The Coronavirus Surface Glycoprotein, p. 73-113. In S. G. Siddell (ed.), The Coronaviridae. Plenum Press, New York.

7. Chambers, P., C. R. Pringle, and A. J. Easton. 1990. Heptad repeat sequences are located adjacent to hydrophobic regions in several types of virus fusion glycoproteins. J Gen Virol 71:3075-80.

8. Chan, D.C., D. Fass, J. M. Berger, and P. S. Kim. 1997. Core structure of gp41 from the HIV envelope glycoprotein. Cell 89:263-73.

9. Chen, C. H., T. J. Matthews, C. B. McDanal, D. P. Bolognesi, and M. L. Greenberg. 1995. A molecular clasp in the human immunodeficiency virus (HIV) type 1 TM protein determines the anti-HIV activity of gp41 derivatives: implication for viral fusion. J Virol 69:3771-7.

10. Chen, J., K. H. Lee, D. A. Steinhauer, D. J. Stevens, J. J. Skehel, and D. C. Wiley. 1998. Structure of the hemagglutinin precursor cleavage site, a determinant of influenza pathogenicity and the origin of the labile conformation. Cell 95:409-17.

11. Chen, J., J. J. Skehel, and D. C. Wiley. 1999. N- and C-terminal residues combine in the fusion-pH influenza hemagglutinin HA(2) subunit to form an N cap that terminates the triple-stranded coiled coil. Proc Natl Acad Sci USA 96:8967-72.

12. Chen, J., S. A. Wharton, W. Weissenhorn, L. J. Calder, F. M. Hughson, J. J. Skehel, and D. C. Wiley. 1995. A soluble domain of the membrane-anchoring chain of influenza virus hemagglutinin (HA2) folds in Escherichia coli into the low-pH-induced conformation. Proc Natl Acad Sci USA 92:12205-9.

13. Chen, L., J. J. Gorman, J. McKimm-Breschkin, L. J. Lawrence, P. A. Tulloch, B. J. Smith, P. M. Colman, and M. C. Lawrence. 2001. The structure of the fusion glycoprotein of Newcastle disease virus suggests a novel paradigm for the molecular mechanism of membrane fusion. Structure (Camb) 9:255-66.

14. Davies, H. A., and M. R. MacNaughton. 1979. Comparison of the morphology of three coronaviruses. Arch Virol 59:25-33.

15. de Groot, R. J., W. Luytjes, M. C. Horzinek, B. A. van der Zeijst, W. J. Spaan, and J. A. Lenstra. 1987. Evidence for a coiled-coil structure in the spike proteins of coronaviruses. J Mol Biol 196:963-6.

16. Delmas, B., and H. Laude. 1990. Assembly of coronavirus spike protein into trimers and its role in epitope expression. J Virol 64:5367-75.

17. Eckert, D. M., and P. S. Kim. 2001. Design of potent inhibitors of HIV-1 entry from the gp41 N-peptide region. Proc Natl Acad Sci USA 98:11187-92.

18. Eckert, D. M., and P. S. Kim. 2001. Mechanisms of viral membrane fusion and its inhibition. Annu Rev Biochem 70:777-810.

19. Fass, D., S. C. Harrison, and P. S. Kim. 1996. Retrovirus envelope domain at 1.7 angstrom resolution. Nat Struct Biol 3:465-9.

20. Furuta, R. A., C. T. Wild, Y. Weng, and C. D. Weiss. 1998. Capture of an early fusion-active conformation of HIV-1 gp41. Nat Struct Biol 5:276-9.

21. Gallagher, T. M. 1997. A role for naturally occurring variation of the murine coronavirus spike protein in stabilizing association with the cellular receptor. J Virol 71:3129-37.

22. Gallagher, T. M., and M. J. Buchmeier. 2001. Coronavirus spike proteins in viral entry and pathogenesis. Virology 279:371-4.

23. Gallagher, T. M., C. Escarmis, and M. J. Buchmeier. 1991. Alteration of the pH dependence of coronavirus-induced cell fusion: effect of mutations in the spike glycoprotein. J Virol 65:1916-28.

24. Gill, S. C., and P. H. von Hippel. 1989. Calculation of protein extinction coefficients from amino acid sequence data. Anal Biochem 182:319-26.

25. Gombold, J. L., S. T. Hingley, and S. R. Weiss. 1993. Fusion-defective mutants of mouse hepatitis virus A59 contain a mutation in the spike protein cleavage signal. J Virol 67:4504-12.

26. Hingley, S. T., I. Leparc-Goffart, and S. R. Weiss. 1998. The spike protein of murine coronavirus mouse hepatitis virus strain A59 is not cleaved in primary glial cells and primary hepatocytes. J Virol 72:1606-9.

27. Holmes, K. V., B. D. Zelus, J. H. Schickli and S. R. Weiss. 2001. Receptor specificity and receptor-induced conformational changes in mouse hepatitis virus spike glycoprotein. Adv Exp Med. Biol 494:173-181.

28. Jiang, S., K. Lin, N. Strick, and A. R. Neurath. 1993. HIV-1 inhibition by a peptide. Nature 365:113.

29. Joshi, S. B., R. E. Dutch, and R. A. Lamb. 1998. A core trimer of the paramyxovirus fusion protein: parallels to influenza virus hemagglutinin and HIV-1 gp41. Virology 248:20-34.

30. Judice, J. K., J. Y. Tom, W. Huang, T. Wrin, J. Vennari, C. J. Petropoulos, and R. S. McDowell. 1997. Inhibition of HIV type 1 infectivity by constrained alpha-helical peptides: implications for the viral fusion mechanism. Proc Natl Acad Sci USA 94:13426-30.

31. Kliger, Y., and Y. Shai. 2000. Inhibition of HIV-1 entry before gp41 folds into its fusion-active conformation. J Mol Biol 295:163-8.

32. Kobe, B., R. J. Center, B. E. Kemp, and P. Poumbourios. 1999. Crystal structure of human T cell leukemia virus type 1 gp21 ectodomain crystallized as a maltose-binding protein chimera reveals structural evolution of retroviral transmembrane proteins. Proc Natl Acad Sci USA 96:4319-24.

33. Krueger, D. K., S. M. Kelly, D. N. Lewicki, R. Ruffolo, and T. M. Gallagher. 2001. Variations in disparate regions of the murine coronavirus spike protein impact the initiation of membrane fusion. J Virol 75:2792-802.

34. Kuo, L., G. J. Godeke, M. J. Raamsman, P. S. Masters, and P. J. Rottier. 2000. Retargeting of coronavirus by substitution of the spike glycoprotein ectodomain: crossing the host cell species barrier. J Virol 74:1393-406.

35. Lambert, D. M., S. Barney, A. L. Lambert, K. Guthrie, R. Medinas, D. E. Davis, T. Bucy, J. Erickson, G. Merutka, and S. R. Petteway, Jr. 1996. Peptides from conserved regions of paramyxovirus fusion (F) proteins are potent inhibitors of viral fusion. Proc Natl Acad Sci USA 93:2186-91.

36. Lescar, J., A. Roussel, M. W. Wien, J. Navaza, S. D. Fuller, G. Wengler, and F. A. Rey. 2001. The Fusion glycoprotein shell of Semliki Forest virus: an icosahedral assembly primed for fusogenic activation at endosomal pH. Cell 105:137-48.

37. Lewicki, D. N., and T. M. Gallagher. 2002. Quaternary structure of coronavirus spikes in complex with carcinoembryonic antigen-related cell adhesion molecule cellular receptors. J Biol Chem 277:19727-34.

38. Lu, M., S. C. Blacklow, and P. S. Kim. 1995. A trimeric structural domain of the HIV-1 transmembrane glycoprotein. Nat Struct Biol 2:1075-82.

39. Luo, Z., A. M. Matthews, and S. R. Weiss. 1999. Amino acid substitutions within the leucine zipper domain of the murine coronavirus spike protein cause defects in oligomerization and the ability to induce cell-to-cell fusion. J Virol 73:8152-9.

40. Luo, Z., and S. R. Weiss. 1998. Roles in cell-to-cell fusion of two conserved hydrophobic regions in the murine coronavirus spike protein. Virology 244:483-94.

41. Malashkevich, V. N., D. C. Chan, C. T. Chutkowski, and P. S. Kim. 1998. Crystal structure of the simian immunodeficiency virus (SIV) gp41 core: conserved helical interactions underlie the broad inhibitory activity of gp41 peptides. Proc Natl Acad Sci U S A 95:9134-9.

42. Malashkevich, V. N., B. J. Schneider, M. L. McNally, M. A. Milhollen, J. X. Pang, and P. S. Kim. 1999. Core structure of the envelope glycoprotein GP2 from Ebola virus at 1.9—A resolution. Proc Natl Acad Sci USA 96:2662-7.

43. Malashkevich, V. N., M. Singh, and P. S. Kim. 2001. The trimer-of-hairpins motif in membrane fusion: Visna virus. Proc Natl Acad Sci USA 98:8502-6.

44. Matsuyama, S., and F. Taguchi. 2002. Communication between S1N330 and a region in S2 of murine coronavirus spike protein is important for virus entry into cells expressing CEACAM1b receptor. Virology 295:160-71.

45. Matsuyama, S., and F. Taguchi. 2002. Receptor-induced conformational changes of murne coronavirus spike protein. J Virol 76:11819-26.

46. Melikyan, G. B., R. M. Markosyan, H. Hemmati, M. K. Delmedico, D. M. Lambert, and F. S. Cohen. 2000. Evidence that the transition of HIV-1 gp41 into a six-helix bundle, not the bundle configuration, induces membrane fusion. J Cell Biol 151:413-23.

47. Munoz-Barroso, I., S. Durell, K. Sakaguchi, E. Appella, and R. Blumenthal. 1998. Dilation of the human immunodeficiency virus-1 envelope glycoprotein fusion pore-revealed by the inhibitory action of a synthetic peptide from gp41. J Cell Biol 140:315-23.

48. Nash, T. C., and M. J. Buchmeier. 1997. Entry of mouse hepatitis virus into cells by endosomal and nonendosomal pathways. Virology 233:1-8.

49. Nehete, P. N., R. B. Arlinghaus, and K. J. Sastry. 1993. Inhibition of human immunodeficiency virus type I infection and syncytium formation in human cells by V3 loop synthetic peptides from gp120. J Virol 67:6841-6.

50. Parry, D. A. 1978. Fibrinogen: A preliminary analysis of the amino acid sequences of the portions of the alpha, beta, and gamma-chains postulated to form the interdomainal link between globular regions of the molecule. J. Mol. Biol. 248:180-189.

51. Rappaport, D., M. Ovadia, and Y. Shai. 1995. A synthetic peptide corresponding to a conserved heptad repeat domain is a potent inhibitor of Sendai virus-cell fusion: an emerging similarity with functional domains of other viruses. Embo J 14:5524-31.

52. Rimsky, L. T., D. C. Shugars, and T. J. Matthews. 1998. Determinants of human immunodeficiency virus type 1 resistance to gp41-derived inhibitory peptides. J Virol 72:986-93.

53. Ruigrok, R. W., A. Aitken, L. J. Calder, S. R. Martin, J. J. Skehel, S. A. Wharton, W. Weis, and D. C. Wiley. 1988. Studies on the structure of the influenza virus haemagglutinin at the pH of membrane fusion. J Gen Virol 69:2785-95.

54. Russell, C. J., T. S. Jardetzky, and R. A. Lamb. 2001. Membrane fusion machines of paramyxoviruses: capture of intermediates of fusion. Embo J 20:4024-34.

55. Schagger, H., and G. von Jagow. 1987. Tricine-sodium dodecyl sulfate-polyacrylamide gel electrophoresis for the separation of proteins in the range from 1 to 100 kDa. Anal Biochem 166:368-79.

56. Siddell, S. G. 1995. The Coronaviridae; an introduction. Plenum Press, New York.

57. Skehel, J. J., and D. C. Wiley. 1998. Coiled coils in both intracellular vesicle and viral membrane fusion. Cell 95:871-4.

58. Slepushkin, V. A., G. V. Kornilaeva, S. M. Andreev, M. V. Sidorova, A. O. Petrukhina, G. R. Matsevich, S. V. Raduk, V. B. Grigoriev, T. V. Makarova, V. V. Lukashov, and Karamov, E. V. 1993. Inhibition of human immunodeficiency virus type 1 (HIV-1) penetration into target cells by synthetic peptides mimicking the N-terminus of the HIV-1 transmembrane glycoprotein. Virology 194:294-301.

59. Stauber, R., M. Pfleiderera, and S. Siddell. 1993. Proteolytic cleavage of the murine coronavirus surface glycoprotein is not required for fusion activity. J Gen Virol 74:183-91.

60. Sturman, L. S., C. S. Ricard, and K. V. Holmes. 1990. Conformational change of the coronavirus peplomer glycoprotein at pH 8.0 and 37 degrees C. correlates with virus aggregation and virus-induced cell fusion. J Virol 64:3042-50.

61. Taguchi, F. 1993. Fusion formation by the uncleaved spike protein of murine coronavirus JHMV variant cl-2. J Virol 67:1195-202.

62. Taguchi, F. 1995. The S2 subunit of the murine coronavirus spike protein is not involved in receptor binding. J Virol 69:7260-3.

63. Tan, K., J. Liu, J. Wang, S. Shen, and M. Lu. 1997. Atomic structure of a thermostable subdomain of HIV-1 gp41. Proc Natl Acad Sci USA 94:12303-8.

64. Vennema, H., G. J. Godeke, J. W. Rossen, W. F. Voorhout, M. C. Horzinek, D. J. Opstelten, and P. J. Rottier. 1996. Nucleocapsid-independent assembly of coronavirus-like particles by co-expression of viral envelope protein genes. Embo J 15:2020-8.

65. Vennema, H., R. Rijnbrand, L. Heijnen, M. C. Horzinek, and W. J. Spaan. 1991. Enhancement of the vaccinia virus/phage T7 RNA polymerase expression system using encephalomyocarditis virus 5′-untranslated region sequences. Gene 108:201-9.

66. Vennema, H., P. J. Rottier, L. Heijnen, G. J. Godeke, M. C. Horzinek, and W. J. Spaan. 1990. Biosynthesis and function of the coronavirus spike protein. Adv Exp Med Biol 276:9-19.

67. Weissenhorn, W., L. J. Calder, S. A. Wharton, J. J. Skehel, and D. C. Wiley. 1998. The central structural feature of the membrane fusion protein subunit from the Ebola virus glycoprotein is a long triple-stranded coiled coil. Proc Natl Acad Sci USA 95:6032-6.

68. Weissenhorn, W., A. Carfil, K. H. Lee, J. J. Skehel, and D. C. Wiley. 1998. Crystal structure of the Ebola virus membrane fusion subunit, GP2, from the envelope glycoprotein ectodomain. Mol Cell 2:605-16.

69. Weissenhorn, W., A. Dessen, S. C. Harrison, J. J. Skehel, and D. C. Wiley. 1997. Atomic structure of the ectodomain from HIV-1 gp41. Nature 387:426-30.

70. Weissenhorn, W., S. A. Wharton, L. J. Calder, P. L. Earl, B. Moss, E. Aliprandis, J. J. Skehel, and D. C. Wiley. 1996. The ectodomain of HIV-1 env subunit gp41 forms a soluble, alpha-helical, rod-like oligomer in the absence of gpl20 and the N-terminal fusion peptide. Embo J 15:1507-14.

71. Westenberg, M., H. Wang, W. F. IJkel, R. W. Goldbach, J. M. Vlak, and D. Zuidema. 2002. Furin is involved in baculovirus envelope fusion protein activation. J Virol 76:178-84.

72. Wild, C., T. Oas, C. McDanal, D. Bolognesi, and T. Matthews. 1992. A synthetic peptide inhibitor of human immunodeficiency virus replication: correlation between solution structure and viral inhibition. Proc Natl Acad Sci USA 89:10537-41.

73. Wild, C. T., D. C. Shugars, T. K. Greenwell, C. B. McDanal, and T. J. Matthews. 1994. Peptides corresponding to a predictive alpha-helical domain of human immunodeficiency virus type 1 gp41 are potent inhibitors of virus infection. Proc Natl Acad SciUSA 91:9770-4.

74. Williams, R. K., G. S. Jiang, and K. V. Holmes. 1991. Receptor for mouse hepatitis virus is a member of the carcinoembryonic antigen family of glycoproteins. Proc Natl Acad Sci USA 88:5533-6.

75. Wilson, I. A., J. J. Skehel, and D. C. Wiley. 1981. Structure of the haemagglutinin membrane glycoprotein of influenza virus at 3 A resolution. Nature 289:366-73.

76. Yang, Z. N., T. C. Mueser, J. Kaufman, S. J. Stahl, P. T. Wingfield, and C. C. Hyde. 1999. The crystal structure of the SIV gp41 ectodomain at 1.47 A resolution. J Struct Biol 126:131-44.

77. Yao, Q., and R. W. Compans. 1996. Peptides corresponding to the heptad repeat sequence of human parainfluenza virus fusion protein are potent inhibitors of virus infection. Virology 223:103-12.

78. Yoo, D. W., M. D. Parker, and L. A. Babiuk. 1991. The S2 subunit of the spike glycoprotein of bovine coronavirus mediates membrane fusion in insect cells. Virology 180:395-9.

79. Young, J. K., R. P. Hicks, G. E. Wright, and T. G. Morrison. 1997. Analysis of a peptide inhibitor of paramyxovirus (NDV) fusion using biological assays, NMR, and molecular modeling. Virology 238:291-304.

80. Zhao, X., M. Singh, V. N. Malashkevich, and P. S. Kim. 2000. Structural characterization of the human respiratory syncytial virus fusion protein core. Proc Natl Acad Sci USA 97:14172-7.

What is claimed is:

1. A method for at least in part inhibiting anti-parallel coiled coil formation of a coronavirus spike protein, said method comprising:
decreasing the contact between heptad repeat regions of the coronavirus spike protein.

2. The method according to claim 1 wherein said decrease is provided by a peptide and/or a functional fragment and/or an equivalent thereof.

3. The method according to claim 2 wherein said decrease is provided by a peptide comprising a heptad repeat region of a corona viral spike protein and/or a functional fragment and/or an equivalent thereof.

4. The method according to any of claims 1-3, wherein said heptad repeat region comprises an amino acid sequence of SARS HR2 according to FIG. 9, and/or a functional fragment and/or an equivalent thereof.

5. The method according to claim 1, wherein said decrease is provided by an antibody and/or a functional fragment and/or an equivalent thereof.

6. The method according to any of claims 1-5, wherein said coronavirus is a group 1 coronavirus.

7. The method according to claim 6, wherein said coronavirus is a feline coronavirus.

8. The method according to claim 7, wherein said feline coronavirus is feline infectious peritonitis (FIP) virus.

9. The method according to claim 6, wherein said coronavirus comprises a human corona virus.

10. The method according to any of claims 1-5, wherein said coronavirus comprises a group 2 coronavirus.

11. The method according to claim 10, wherein said coronavirus comprises a mouse hepatitis virus (MHV).

12. The method according to any of claims 1-5, wherein said coronavirus causes Severe Acute Respiratory Syndrome (SARS).

13. A method for inhibiting coronavirus spike protein mediated cell to cell fusion, said method comprising:
decreasing the contact between said spike protein's heptad repeat regions.

14. A method of selecting a binding compound to a heptad repeat region of a coronavirus spike protein, said method comprising:
contacting in vitro at least one heptad region of a coronavirus spike protein with a collection of compounds and measuring the formation of an anti-parallel coiled coil in said protein.

15. A binding compound selected by the method according to claim 14.

16. An antibody or a functional fragment and/or equivalent thereof, said antibody or functional fragment and/or equivalent thereof capable of decreasing the contact between heptad repeat regions of a coronavirus spike protein.

17. A pharmaceutical composition comprising:
the binding compound of claim 15, and/or
an antibody or functional fragment and/or equivalent thereof capable of decreasing the contact between heptad repeat regions of a coronavirus spike protein, and a suitable diluent and/or carrier.

18. A method of treating coronavirus infections, said method comprising:
providing to a subject the pharmaceutical: composition of claim 17.

19. A diagnostic kit for detecting coronavirus infection in a sample taken from a subject, said diagnostic kit comprising:
the binding compound of claim 15 or
an antibody or functional fragment and/or equivalent thereof capable of decreasing the contact between heptad repeat regions of a coronavirus spike protein, and
means for detecting binding of said compound or antibody to said coronavirus.

20. A diagnostic kit for detecting coronavirus antibodies in a sample taken from a subject, said diagnostic kit comprising:
the binding compound of claim 15, and
means for detecting binding of said compound to said antibodies.

21. A method of attenuating a coronavirus, said method comprising:
decreasing the contact between heptad repeat regions of the spike protein of said coronavirus.

22. An attenuated coronavirus characterized in that the contact between heptad repeat regions of the spike protein of said coronavirus is decreased.


SEQUENCE LISTING (28 sequences)

SEQ ID NO: 1 - DNA, 42 bp, Artificial
  Primer 973
gtggatccatcgaaggtcgtcaatatagaattaatggtttag

SEQ ID NO: 2 - DNA, 37 bp, Artificial
  Primer 974
gtggatccatcgaaggtcgtaatgcaaatgctgaagc

SEQ ID NO: 3 - DNA, 29 bp, Artificial
  Primer 975
ggaattcaattaataagacgatctatctg

SEQ ID NO: 4 - DNA, 27 bp, Artificial
  Primer 976
cgaattcattccttgaggttgatgtag

SEQ ID NO: 5 - DNA, 38 bp, Artificial
  Primer 990
gcggatccatcgaaggtcgtgatttatctctcgatttc

SEQ ID NO: 6 - DNA, 25 bp, Artificial
  Primer 1151
gtggatccaaccaaaagatgattgc

SEQ ID NO: 7 - DNA, 28 bp, Artificial
  Primer 1152
ggaattcaattgagtgcttcagcatttg

SEQ ID NO: 8 - protein, 102 aa, Mouse Hepatitis Virus
  MISC_FEATURE (1)..(102): Amino acids 947 to 1048, corresponding to heptad repeat region one (HR1)
PFSLSVQYRINGLGVTMNVLSENQKMIASAFNNALGAIQDGFDATNSALGKIQSVVNANAEALNNLLNQLSNRFGAISASLQEILTRLEAVEAKAQIDRLIN

SEQ ID NO: 9 - protein, 102 aa, Human Coronavirus strain OC43
  MISC_FEATURE (1)..(102): Amino acids 981 to 1082, corresponding to heptad repeat region one (HR1)
PFYLNVQYRINGLGVTMDVLSQNQKLIANAFNNALYAIQEGFDATNSALVKIQAVVNANAEALNNLLQQLSNRFGAISASLQEILSRLDALEAEAQIDRLIN

SEQ ID NO: 10 - protein, 116 aa, Human Coronavirus strain 229E
  MISC_FEATURE (1)..(116): Amino acids 768 to 883, corresponding to the HR1 region
PFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLIT

SEQ ID NO: 11 - protein, 116 aa, Feline Infectious Peritonitis virus strain 79-1146
  MISC_FEATURE (1)..(116): Amino acids 1041 to 1156, corresponding to the HR1 region
PFAVAVQARLNYVALQTDVLNKNQQILANAFNQAIGNITQAFGKVNDAIHQTSQGLATVAKALAKVQDVVNTQGQALSHLTVQLQNNFQAISSSISDIYNRLDELSADAQVDRLIT

SEQ ID NO: 12 - protein, 102 aa, infectious bronchitis virus strain Beaudette
  MISC_FEATURE (1)..(102): Amino acids 770 to 871, corresponding to the HR1 region
PFATQLQARINHLGITQSLLLKNQEKIAASFNKAIGHMQEGFRSTSLALQQIQDVVSKQSAILTETMASLNKNFGAISSVIQEIYQQFDAIQANAQVDRLIT

SEQ ID NO: 13 - protein, 102 aa, SARS-associated coronavirus
  MISC_FEATURE (1)..(6): amino acids derived from the proteolytic cleavage site of the GST-fusion protein
GSIEGRQYRINGLGVTMNVLSENQKMIASAFNNALGAIQDGFDATNSALGKIQSVVNANAEALNNLLNQLSNRFGAISASLQEILTRLEAVEAKAQIDRLIN

SEQ ID NO: 14 - protein, 82 aa, SARS-associated coronavirus
  MISC_FEATURE (1)..(2): amino acids derived from the proteolytic cleavage site of the GST-fusion protein
GSNQKMIASAFNNALGAIQDGFDATNSALGKIQSVVNANAEALNNLLNQLSNRFGAISASLQEILTRLEAVEAKAQIDRLIN

SEQ ID NO: 15 - protein, 52 aa, SARS-associated coronavirus
  MISC_FEATURE (1)..(6): amino acids derived from the proteolytic cleavage site of the GST-fusion protein
GSIEGRNANAEALNNLLNQLSNRFGAISASLQEILTRLEAVEAKAQIDRLIN

SEQ ID NO: 16 - protein, 49 aa, SARS-associated coronavirus
  MISC_FEATURE (1)..(2): amino acids derived from the proteolytic cleavage site of the GST-fusion protein
GSNQKMIASAFNNALGAIQDGFDATNSALGKIQSVVNANAEALNNLLNQ

SEQ ID NO: 17 - protein, 48 aa, Mouse Hepatitis Virus
  MISC_FEATURE (1)..(48): Amino acids 1215 to 1262 from mouse hepatitis virus, corresponding to heptad repeat region two
PDLSLDFEKLNVTLLDLTYEMNRIQDAIKKLNESYINLKEVGTYEMYV

SEQ ID NO: 18 - protein, 46 aa, Human Coronavirus strain OC43
  MISC_FEATURE (1)..(46): Amino acids 1249 to 1294 from human corona virus strain OC43, corresponding to heptad repeat region two
PDLSLDYINVTFLDLQVEMNRLQEAIKVLNQSYINLKDIGTYEYYV

SEQ ID NO: 19 - protein, 60 aa, Human Coronavirus strain 229E
  MISC_FEATURE (1)..(60): Amino acids 1053 to 1112 from human corona virus strain 229E, corresponding to heptad repeat region two
PDLVVEQYNQTILNLTSEISTLENKSAELNYTVQKLQTLIDNINSTLVDLKWLNRVETYI

SEQ ID NO: 20 - protein, 60 aa, Feline Infectious Peritonitis virus strain 79-1146
  MISC_FEATURE (1)..(60): Amino acids from feline peritonitis virus, corresponding to heptad repeat region two
PEFTLDIFNATYLNLTGEIDDLEFRSEKLHNTTVELAILIDNINNTLVNLEWLNRIETYV

SEQ ID NO: 21 - protein, 46 aa, infectious bronchitis virus strain Beaudette
  misc_feature (1)..(46): Amino acids 1045 to 1090 from Beaudette strain, corresponding to heptad repeat region two
PDFDKFNYTVPILDIDSEIDRIQGVIQGLNDSLIDLEKLSILKTYI

SEQ ID NO: 22 - protein, 45 aa, SARS-associated coronavirus
  MISC_FEATURE (1)..(6): amino acids derived from the proteolytic cleavage site of the GST-fusion protein
GSIEGRDLSLDFEKLNVTLLDLTYEMNRIQDAIKKLNESYINLKE

SEQ ID NO: 23 - protein, 336 aa, SARS-associated coronavirus
  MISC_FEATURE (1)..(336): GST-HR1 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSQARLNYVALQTDVLNKNQQILANAFNQAIGNITQAFGKVNDAIHQTSQGLATVAKALAKVQDVVNTQGQALSHLTVQLQNNFQAISSSISDIYNRLDELSADAQVDRLIT

SEQ ID NO: 24 - protein, 277 aa, SARS-associated coronavirus
  MISC_FEATURE (1)..(227): GST-HR2 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEFTLDIFNATYLNLTGEIDDLEFRSEKLHNTTVELAILIDNINNTLVNLEW

SEQ ID NO: 25 - DNA, 582 bp, SARS-associated coronavirus
  CDS (1)..(582): Protein sequence derived from RT-PCR fragment
gaaatchcgscttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatatt

SEQ ID NO: 26 - protein, 194 aa, SARS-associated coronavirus
  misc_feature (3)..(3): The ′Xaa′ at location 3 stands for Thr, Pro, or Ser.
EIXXSANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYI

SEQ ID NO: 27 - protein, 49 aa, SARS-associated coronavirus
  MISC_FEATURE (1)..(49): Amino acids 146 to 194, corresponding to heptad repeat region two from SARS
PDVDLGDISGINASVVNIQKEIDRLNEVAKNLNESLIDLQELGKYEQYI

SEQ ID NO: 28 - protein, 60 aa, Feline Infectious Peritonitis virus strain 79-1146; identical to SEQ ID NO: 20
  MISC_FEATURE (1331)..(1390): Heptad repeat region 2 sequence